SEQUENCE LISTING (58 sequences)

SEQ ID NO: 1 - DNA, 3486 bp, Komagataella phaffii
  source 1..3486: mol_type = other DNA
atgcgagaccgcggccgcatgagcaatctacccccaacttttggttccactagacaatctccagaagaccaatcacctcccgtgcccaaggagctgtcattcaatgggaccacaccctcaggaaagctacgcttatttgtctgtcagacatgtactcgagcatttgctcgtcaggaacacttgaaacgacacgaaaggtctcacaccaaggagaaacctttcagctgcggcatttgttctcgtaaattcagccgtcgagatctgttattgagacatgcccaaaaactgcacagcaactgctctgatgcggccataacaagactaaggcgcaaggcaactcgtcggtcttctaatgccgcgggttccatatctggttctactccggtgacaacgccaaatactatgggtacgcccgaagatggcgagaaacgaaaagttcagaaactggccggccgccgggactcaaatgaacagaaactgcaactgcaacaacaacatctacagcaacaaccacagttgcaataccaacaatctcttaagcagcatgaaaatcaagtccagcagcctgatcaagatccattgatatccccgagaatgcaattattcaatgattccaaccatcacgtaaacaatttgtttgatcttggactaagaagagcttccttctccgccgttagtggaaataattatgcccattatgtgaataattttcaacaagatgcctcttctaccaatccaaatcaagattcaaataatgccgaatttgagaatattgaattttctaccccacaaatgatgcccgttgaagatgctgaaacttggatgaacaacatgggtccaattccgaacttctctctcgatgtgaacaggaacattggtgatagctttacagatatacaacacaagaattcagagcctattatatccgaaccgcccaaggacaccgctccaaacgacaagaagttgaatggctactctttttacgaagcccccatcaagccattagaatccctattttctgtcaggaatacaaagagaaacaagtataaaacaaatgacgactctccagacaccgtggataataactccgcaccggctgctaataccattcaagaacttgagtcttctttgaatgcatccaagaatttttgcttgccaactggttattccttctatggtaatttggaccaacagactttctctaacacgttatcatgcacttcttctaatgccacaatttcgcccattctactcgataactccattaataataactccactagtgacgtgagaccagaatttagaacacaaagtgtcacctctgaaatgagtcaagcccctccccctcctcaaaaaaacaactcgaaatattccaccgaagttctttttaccagcaacatgcggtcgtttattcactacgctctttccaagtatccttttattggtgtgcccactccaactcttccggagaacgaaagactaaatgaatatgctgattcattcaccaaccgtttcttaaatcattatcctttcatacatgtcacgattctcaaagaatactcccttttcaaggcaattttagatgagaatgagtcgactaagaactgggaaaataatcagttttacttagagaaccaacgaatatcaattgtttgtcttcctcttttggtggctacgataggtgcagtactatcaaacaacaaaaaggatgcttcgaatttatacgaagcttcaaggcgttgtattcatgtttacttagattccaggaaaaagatacccacttccttgtccgcaaataacaatgactctccactttggctaattcaatccctgacgttatctgttatgtatgggttatttgcggacaatgacattagtttgaatgtcgtgatcagacaagttaacgcacttaattctctggtcaagacttcgggcctgaataggacctcaattatagatcttttcaacatcaacaaacctttggataatgaactctggaatcaattcgtgaaaatagagtccaccgtaaggacaatccacacgatttttcaaatcagttccaacttaagcgccttgtacaatattattccatcgttgaaaattgatgacctaatgattactctaccagttcccacaacactttggcaagctgattcttttgtgaaattcaaaagtctaagttacggaaatcagatcccttttcaatatacaagagtactacagaatttgattgattacaatcagccattgagcgatggaaaatttttgtatgaaaaccatgtaagtgagtttggactcatatgcctacagaatggtctacaccaatacagctatttccaaaaattgactgctgtcaataacagagaagatgcgctattcacaaaggttgttaattcacttcacagttgggataggatgatttcgaattctgatttgtttccaaagaagatatatcagcagagttgcttgattttggactcaaagttgcttaataatttcctgattgtcaagagctcattgaaagtttcgaccggagacgttagttctttgaataagttaaaagaaaacgtgtggcttaaaaactggaatcaagtgtgtgctatctattataacagcttcatgaacattcctgctcccagtattcaaaagaagtacaatgacatagagtttgtggatgacatgattaatttgagtctaatcatcatcaagattatgaaactcattttctataacaatgtcaaagacaattatgaggatgaaaatgacttcaaattgcaagagttaaatttaacatttgacaattttgatgagaaaatatccttgaatttgacaatattattcgatatatttttgatgatctacaagataattaccaattacgaaaagtttatgaagatcaaacacaagtttaattactacaattctaattcgaatataagcttcttgcatcatttcgaactctcctcggttatcaataacacccaaatgaaccagaatgattatatgaaaacagatattgatgaaaagcttgatcagcttttccacatctatcaaacatttttccggctgtatctggatttagaaaagtttatgaagttcaaattcaactatcatgactttgagacagagttttcaagtctctcaatatccaatatactgaacactcatgctgcttctaacaatgacacaaatgctgctgatgctatgaatgccaaggatgaaaaaatatctcccacaactttgaatagcgtattacttgctgatgaaggaaatgaaaattccggtcgtaataacgattcagaccgcctgttcatgctgaacgagctaattaattttgaagtaggtttgaaatttctcaagataggtgagtcattttttgatttcttgtatgagaataactacaagttcatccacttcaaaaacttaaatgacggaatgttccacatcaggatatacctagaaaaccgactagatggtggtgtctag

SEQ ID NO: 2 - protein, 1161 aa, Komagataella phaffii
MRDRGRMSNLPPTFGSTRQSPEDQSPPVPKELSFNGTTPSGKLRLFVCQTCTRAFARQEHLKRHERSHTKEKPFSCGICSRKFSRRDLLLRHAQKLHSNCSDAAITRLRRKATRRSSNAAGSISGSTPVTTPNTMGTPEDGEKRKVQKLAGRRDSNEQKLQLQQQHLQQQPQLQYQQSLKQHENQVQQPDQDPLISPRMQLFNDSNHHVNNLFDLGLRRASFSAVSGNNYAHYVNNFQQDASSTNPNQDSNNAEFENIEFSTPQMMPVEDAETWMNNMGPIPNFSLDVNRNIGDSFTDIQHKNSEPIISEPPKDTAPNDKKLNGYSFYEAPIKPLESLFSVRNTKRNKYKTNDDSPDTVDNNSAPAANTIQELESSLNASKNFCLPTGYSFYGNLDQQTFSNTLSCTSSNATISPILLDNSINNNSTSDVRPEFRTQSVTSEMSQAPPPPQKNNSKYSTEVLFTSNMRSFIHYALSKYPFIGVPTPTLPENERLNEYADSFTNRFLNHYPFIHVTILKEYSLFKAILDENESTKNWENNQFYLENQRISIVCLPLLVATIGAVLSNNKKDASNLYEASRRCIHVYLDSRKKIPTSLSANNNDSPLWLIQSLTLSVMYGLFADNDISLNVVIRQVNALNSLVKTSGLNRTSIIDLFNINKPLDNELWNQFVKIESTVRTIHTIFQISSNLSALYNIIPSLKIDDLMITLPVPTTLWQADSFVKFKSLSYGNQIPFQYTRVLQNLIDYNQPLSDGKFLYENHVSEFGLICLQNGLHQYSYFQKLTAVNNREDALFTKVVNSLHSWDRMISNSDLFPKKIYQQSCLILDSKLLNNFLIVKSSLKVSTGDVSSLNKLKENVWLKNWNQVCAIYYNSFMNIPAPSIQKKYNDIEFVDDMINLSLIIIKIMKLIFYNNVKDNYEDENDFKLQELNLTFDNFDEKISLNLTILFDIFLMIYKIITNYEKFMKIKHKFNYYNSNSNISFLHHFELSSVINNTQMNQNDYMKTDIDEKLDQLFHIYQTFFRLYLDLEKFMKFKFNYHDFETEFSSLSISNILNTHAASNNDTNAADAMNAKDEKISPTTLNSVLLADEGNENSGRNNDSDRLFMLNELINFEVGLKFLKIGESFFDFLYENNYKFIHFKNLNDGMFHIRIYLENRLDGGV

SEQ ID NO: 3 - DNA, 438 bp, synthetic construct
  source 1..438: mol_type = other DNA
atgggtgctttcaccgagaagcaggaagcacttgtttcctcttcgttcgaagcttttaaggctaacatccctcaatactctgttgtgttttacacgtccattctagaaaaagctcctgctgccaaggacctcttctcttttctgtccaacggtgtagatccatccaatcccaaattaacaggtcacgctgagaaattgttcggtttagtcagagatagcgctggacaattgaaagcaaatggtactgtggttgctgatgctgccttgggcagcatccatgcacagaaggcaattacagacccacaatttgttgttgtgaaggaagctctgcttaaaactataaaggaagccgtcggagacaaatggagtgacgagttgtcatcagcttgggaggtagcttatgatgagttggccgcagcaatcaaaaaggcattctaa

SEQ ID NO: 4 - protein, 145 aa, synthetic construct
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 5 - DNA, 438 bp, synthetic construct
  source 1..438: mol_type = other DNA
atgggtgcatttacagaaaaacaagaggctttagtatcctcatcttttgaagctttcaaagccaatattcctcaatactccgttgttttctatacgtccattttggaaaaggctccagcagctaaggaccttttctctttcttgtcgaacggcgtggatccctcaaatcctaagctgactggtcacgccgagaagctttttggtttggtcagagacagcgccggacagctgaaagctaacggtacagttgtggcagatgctgccttgggatctatacatgcacaaaaggctatcaccgacccacagtttgtggttgtaaaagaggctctactcaaaactatcaaggaagcagttggtgacaaatggagcgatgaattgtccagtgcatgggaggtcgcttacgatgagttagctgctgcaatcaaaaaggctttctaa

SEQ ID NO: 6 - protein, 145 aa, synthetic construct; identical to SEQ ID NO: 4

SEQ ID NO: 7 - DNA, 939 bp, Pichia pastoris
  source 1..939: mol_type = other DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 8 - DNA, 558 bp, Pichia pastoris
  source 1..558: mol_type = other DNA
cgactattatcgatcaatgaaatccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacg

SEQ ID NO: 9 - DNA, 822 bp, Pichia pastoris
  source 1..822: mol_type = other DNA
caggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 10 - DNA, 424 bp, Pichia pastoris
  source 1..424: mol_type = other DNA
ataactgtcgcctcttttatctgccgcactgcatgaggtgtccccttagtgggaaagagtactgagccaaccctggaggacagcaagggaaaaatacctacaacttgcttcataatggtcgtaaaaacaatccttgtcggatataagtgttgtagactgtcccttatcctctgcgatgttcttcctctcaaagtttgcgatttctctctatcagaattgccatcaagagactcaggactaatttcgcagtcccacacgcactcgtacatgattggctgaaatttccctaaagaatttctttttcacgaaaattttttttttacacaagattttcagcagatataaaatggagagcaggacctccgctgtgactcttcttttttttcttttattctcactacatacattttagttattcgccaac

SEQ ID NO: 11 - DNA, 1683 bp, Pichia pastoris
  source 1..1683: mol_type = other DNA
atggagtttgtcgcccgtcagtccatgaatgcctgtccctttgtcaggtcaacttctacccaccatttgaagaagttggcagcaaacagttctctagctgctactgctagtcattgtcccgtggttggccctgctctccaacagcagagatactactctcaaccttccaagccagcccaagcccaaacctccgacattgctactgggatcaagaaggatgtttctccgatccgtatggactctaatgaaaccgcctttgattacaatggaatgtatgagtctgatcttgcgaataaacgtaaagataactcgtatcgttatttcaataacatcaaccgtctagccaaggagtttcccaaggcacatcgccagaccgaagatgacaaggtgaccgtctggtgctctaacgactacttaggaatgggtaggcatcctgagattatcaaaaccatgaaggctaccatggacaagtacggttccggagcaggaggaactaggaacattgcaggtcataaccacgccgctatcaatttggaaagcgagttggcttgcttgaacaagaaggaagcggctctggtgttttcatcatgtttcatagctaacgatgcaatcatctcgttgttgggacaaaaaatcaaaaatttggtcattttctctgaccagtcgaatcatgcttccatgatattgggtgtgcgtaactccaaagcgaagaagcacatcttcaagcacaacaatttgaaggatctggagtcgcagttagctcagtaccccaagtcgactcctaaactgatcgccttcgagtcagtttactctatgtgtggatctgtggctcccattgagaagatttgcgatttggctaaaaggtacggtgccctcaccttcttggatgaagttcatgctgttggaatgtatggtcctcatggacagggtgtagctgagcatttggactttgatctgcatttacagtctggaatcgccagtcctagcgtggtggacaaacgcaccatattggatcgtgtcgacatgattactggtacttgcggaaagtcatttggtactgttggaggttacgttgctggtagtgccaacctaattgattggttaagatcctatgcgccaggtttcattttcactaccacacttcctcctgctatcatggctggtacagccacttctgttcgtattgttagggccgacattgaggcccgtatcaagcaacagcttaatactcgctacgtcaaagactcatttgaaaaccttggtattccagtcattccaaacccaagtcacattgttcctgttctagttggaaatgctgcagatgccaagaaggcatccgatatgttaatgaacaaacaccgtatttatgttcaagctattaactaccctactgtgcctgtcggtgaagaacgactaaggattactcctactccaggtcatggaaaggagatttgtgaccagctgatcagcgctgtcgacgatgtttttactgagcttaatttaccaagaatcaacaaatggcagtcccaaggtggtcattgcggtgttggtgatgctaattacgtaccagaacccaatctgtggactcaggaccagctcagcttgacaaaccaagacttgcactccaatgtgcacaacccagtgattgagcagatcgaaacctcatcaggagtcagattgtag

SEQ ID NO: 12 - DNA, 1026 bp, Pichia pastoris
  source 1..1026: mol_type = other DNA
atggtgcataaggctgaatacttggacgaccacccaactcagatttccagcattctttcaggaggttacaaccacccattacttcgtgaatggcaacatgaacgtcaactcaacaaaaacatgttcatctttcccctgtttgtcacagatcgaccagacgaagaagaacttattcctagtctacctaatatcaagaggtttggcgttaacaagttgattccttatgtaggaggtttggtttccaaaggattgagggcggtgatcctatttggtgttcctctgaagcccggtgtgaaagatgaagaaggaacggccgctgatgatccagagggacctgttatccaagccatcaaacacttgagaaagaactttcctgacctgtatatcatcaccgatgtctgtctatgtgagtacaccagccatggacattgtggaatactatatgaggatggcactatcaacagagagctctcagtccgtcgtattgctgctgtagctgtcaaatatgctcaagctggagccaactctgtggctccttctgatatgactgacggcagaataagagatattaaagaaggcttactaagtgcaggactggcacataaaacgtttgttatgtcctacgctgcaaaattctctggtaatttgtatggccctttcagagatgctgcaggttcctgtccatctcaaggggacagaaaatgttaccagcttccttctggaggaaaagggttggcccatcgtgctctgattcgtgatatgaatgaaggcactgatggaattattgtcaaaccatctacattctatttggacattgtcgctgatgcttatcagctttgtaaagactatcctatctgctgttaccaggtttctggagagtacgccatgctacatgcagcggcagagaagaatattgttgatctgaaatcaatcgcttttgaagctcatcaaggattcttgcgggctggagctcgtttaatcattagttactttacccctgaattcctggagtggttatctgaatga

SEQ ID NO: 13 - DNA, 1035 bp, Pichia pastoris
  source 1..1035: mol_type = other DNA
atgaaccaaatcgaacagagcggacccattgattgcagttccttgaaattggggtcccgaaagtccgctctggctataatccaggcagaaatcgtccgccaattgatattgaaagaataccctgaattggagacgaagttggtcagtgtgtccaccctgggggaccaagtccagaataaagcacttttcacgtttggaggaaaatctttgtggaccaaagaacttgagatgttgttgttggagagtgtgggaggatttgaccaaatagacatgattgtacactcgttgaaagacatgccaactcatttaccagacgaatttgagctgggttgcattattgaaagagaagaccctagagacgctttggtcgtgcaagatggtttatcttacaagtcattggccgaccttccagagggagctgtggtcggtacgtcttcggttagaagatcggctcaactactgaagaatttccctcatctgaaattcaaatctgttagaggaaaccttcagaccagactaagaaaattagatgatccagattccgagtactgctgtctcctccttgcagcagccggtttaatcaggacaggcttacaacacagaatttcaatgtatttgaacgacgatgtgatgtaccactccgtcggacaaggagcattaggagtagagatcagaaaaggtgaccaattcatgaaaaatatctgtgaaaagattgggcatagaaccaccacccttcgttgtcttgcagagagagcactgctgagatatctagagggaggctgctcggtgccaattggggtctccactatttatagcgaggatacgaaggaacttaccatgaactccctagtcgtcagttgtaacggtcgtgactcggtaacagaatcaatgactgaagtcgtgactactgaagagcaagctgaagatttcggtgaaaggctggcccagaagctcatagatcaaggtgcgaaacgcattcttgacgagatcaacttcaacaagatcaaagagattaaggaagagggtttacattaa

SEQ ID NO: 14 - DNA, 798 bp, Pichia pastoris
  source 1..798: mol_type = other DNA
atgccaaaagccattcttctgaagaataaaactacaccgaaggatccttatctggagaacttcgtaagtagtggctactcgaccgatttcgtaccacttttagatcatattcacatggagaaatctgagatcatcgcatttctcaagactgactactttttgcataaaactttggcgtttattattacgtcccaaagagctgtagaaatgctgaatgagtgtatgcaaatactgagacgtactgatcctgaaattacacaaatcatctatagtaaacctgtctatacagttggccctgccacctacagaatacttgcggatgctggcttcgtggatctacgaggcggagataaggcaggaaacggatccattctagcccagataattttgaatgatgacatttacactggaattgaagattctgacaagcatataacgtttttcacgggagaaacaaggagagacataattcccaaatgtttactctctaacaactttcaactttacgaaaagattgtctacaagactcttcctagggatgatatcgtgactagattcaagtctgccgttgacagcatcgaccaatcgcaaagaagttccagttgggtggtcttcttttcgcctcaaggaacagaggacattgtaacgtatcttcaacacaccaaagaccaatttaatattgcatctatcgggccaaccacagaaaaataccttctaagcaaaaacctgaaaccaaaagttgtggcacctaagccagagcctatctctttactattgtctatacaaaaagtgcactaa

SEQ ID NO: 15 - DNA, 1086 bp, Pichia pastoris
  source 1..1086: mol_type = other DNA
atgagtagatttccagaactgaagaatgaccttattttaagggcagctcgtggtgaaaaagttgaacgtcccccaatatggattatgagacaggccggaagatatcttccggagtaccatgaggtcaaaggaggtagggacttctttgaaacttgcagggatgctgagattgcttctgaaattactatccagccgattacgcattttgacggtctgatcgatgcagctattatcttcagtgatatcttggtgattcctcaagctatgggcatggaagttaagatggtggacaaagttggcccacagttccccaatccgctaagaaaaccgtctgacttggatcatttgaaaaaagacgttgacgttttgaaggaactcgattgggccttcaaagctatctcattgaccagaaaaaaactcaatggacgagtgcctttgcttggattttgtggtgctccttggactctactggtttatatgactgaaggaggcggtaccaagatgtttcgatttgcaaaagagtggatctacaagtttaccaaggaatctcatcaattactccaacagatcactgacgttgcagttgaattcttagctcagcaagttgttgcaggtgcccaaatgttacaagtttttgaatcttggggcggtgaattggggcctgatgaattcgatgagttttctttgccttatttgagacagatttcctctaaacttcccctgaggttgaaggaacttggaatcacagagaatgttcccataactgtctttgctaaaggctcttggtacgccttggagcaattgtgcgacagtggttatgatgttgtctcgttggattggttattccgtccaagtgatgctgtccagattgctaacggaagaatcgcattgcaaggtaatcttgaccctggaaccatgtacggctccaaagaaaccatttccaagaaagtggacaaaatgatcaagggttttggtggaggaaagcaaaactacataattaattttggacacggcactcatccattcatggatccagaacagatcagatggttcttacaagaatgtcatcgcattggatctcaatag

SEQ ID NO: 16 - DNA, 978 bp, Pichia pastoris
  source 1..978: mol_type = other DNA
atggccatcgactctgatatcaatctaagctctcccaatgattccatccgtcaaaggatgttcgagcttatccagcggaagcaactcgaaattgtcgctgcattggaggcaattgaaggaaacgataccaaatttcgttctgattcttgggaaagaggagccgaaggtggaggaggaagatctatgcttattcaagatggaagagtgtttgaaaaggctggtgtaaatatttccaaggttcatggcgtattgcctcctcaagctgtgagccagatgagaaatgaccactccaagctagatctgcctgcgggaacctctctgaagttctttgcctgtgggctttcgttggtcattcatccccataatccccatgctccaactacccatctgaattatcgctacttcgaaacttgggatgaaactggaaagcctcacacctggtggtttgggggcggtgctgatttaacgccttcgtacctgtatcccgaggatgccaagcaattccatcaagcccataaggatgccctggacaaacacgatgttagcttgtacccgagattcaaaaagtggtgtgatgaatactttctgatcaaacatcgaaatgaaactagaggtattgggggtattttctttgatgattttgacgagtttgatgctgagaggtccctgaagttggttgaagattgtttcaatgctttcttggaatcttatcccgctatcactcgaaaaaggatggacaccccttcaactgatgctgagaagaactggcaacaaattagaagaggaagatatgtcgaattcaacttagtattggatagaggtactcaatttggtttgagaacgcctggatctcgtgttgaaagtattttgatgtcgttgccaagaacagctggttgggtctatgatcatcatccagagcctggctccagagaagaggagttattgcaggtactacaaaatcctattgaatgggtatga

SEQ ID NO: 17 - DNA, 1683 bp, Pichia pastoris
  source 1..1683: mol_type = other DNA
atgctgaaaagtcttgcaccaaattcctcaattgccgttttaggttcagggatatctggattgactttcagcttttttttgaatcggttgcgtcccgatgttaagatccatatctttgaaaaatccaagcaggttggaggatggatcagatcagaagagcatgaaacttttcattttgaaaagggacccagaactttgagaggcacaaatacgggtaccttgatgttgttggatcttcttaccaagataggagcaaatgacaaggtcctgggactgcacaaagattctcttgctaataaaaagtatctgttgtccccgttctcagatgttcacggaaacaacgcaaagcttcttcaagtgccacaggatttcagctcttttgtaaagttcatgtttgacccgttgtctaaggatctcattctcggtcttttgaaagaaccatggcaaccaaaattaaagtattcagatgagtcggttgaccattttttcaacagaagatttgctaccaaactatcagagaatatcgtcagcgcaattgtgcatggaatctatgcgggcgacgtgaagaagttaagtgtgaaagccatcttccctaggctccctgagatggaacaggaaagtggctctattataaggtatatgatcgcccaatacaggacaaaaaagaacgtcaaacaaaaagttgacccttttttggcagattatgaaaaattgatcggtacatctttgagtttcaaaaatatttctttgtttctgaaaaactttcccatgctgagttttcagggtggactacagaaacttcccatctcattgaagaaccatttatcacagattgaaaacatcaagtttcattttgacagcaaaatcaaaaacattgctttggagagcggtaaggtggcattgactgaccatgatcaggtttatcttgttgaccatgtgagatctaccattaataccaacgaattggccaaaatcatttcacccgttgttccaagttctactaagaaaaaatccgttttcaaatccaaagcgaatggcccagggctggtcaaatgtttgagctggctacactatacaaatatactaatgtgcaacatttatatacctaagcacgtctcaaaatctatcaccggatttggatacttggttcctcgatcaatgtcttctcaggcatccaaacttctcggtgtcatatttgactcagacatcgagactgcaatgactcctaattttacagaggccaacattacggcgataaacagtaactctgcatctcccaagcaactccaaaagttttctgaccaattcgtcaataatgatctccctaaatacaccaagttgacgctaatgcttggaggtcattatctcaagtcggaggcagacatgcccggttccgcagagagtaaacatgctgtcaaggcgattctgtcaaatcacctgaatattgatctagatgagtttgcatctttgccagacttcaagatggaaatcaccaagatccccaactgcattccccaatatgaagttgggtatcttgatctcaagagaaaggttcagaatgcagcctccaaagagttcaacgaccaaataagttttggaggcatggcatttggtgatggtgtggggatccctgactgtgtccagaatgcattcaaagattcggctaccctcagtggcatttaa

SEQ ID NO: 18 - DNA, 1125 bp, Pichia pastoris
  source 1..1125: mol_type = other DNA
atgcttaaccgtcgtttccaatctaccgtgtcctcgagtctgaacaagggcactggaatagtgttcatgaatatgggtggtccctccactgtcaaggaaacctatgactttttatttcgtcttttctcggacggagatttaatcccgtttggcagatttcagaacatcctggcccgcttcattgcaagtagaagaacacccaaaattgaatcctactacaaagctatcggaggtgggtctcctatccgaaagtggtctgaataccagagttctaaactatgtgaaaaattagacattatcagtccacaatcggctcctcataagccttatgttgccttcagatacgctaatcctctcactgaagatactttacaaaagatgaaaaatgatggaattactaaggccattgccttttctcaatatccgcaatttagttattcaaccaccggatcatcgattaacgaactttacaggcaatcgaaaattttggaccctgatcaatctattaaatggacagttatagatcgctggcctgaccacccagccttagttaaaactttcgcagctcatatcaaagatactctaaacagattcaaaactgaaaatggactgactgacacaaaagacgtcgtcctccaattcagtgctcattctttaccaatggatattgtcaataaaggagattcgtatcctgcagaagtcgcagcgagtgtctttgccattatgaaagaacttaacttctcaaatccttataaattaacctggcaatcacaggttggcccaaagccttggctgggtgctcaaactgaaaaaattaccaagcagctagcatccagtgatgttcctggagtcgttttggttcctattgcctttacctctgatcatattgaaactctccatgaactggatattgaactgattcaagaactacctaatccttcaaaagtaaagcgagttgaatcgttgaacggagaccaaactttcattgactccttggcagaactagtgaagagtcacattgattcgaaggttgtattttccaaccagttgccattggattccatgctgggagtagtgtcagataattccctcacagatccaaaagagtttttcagagcccattga

SEQ ID NO: 19 - DNA, 30 bp, synthetic construct
  source 1..30: mol_type = other DNA
gagggtctcggatggagtttgtcgcccgtc

SEQ ID NO: 20 - DNA, 34 bp, synthetic construct
  source 1..34: mol_type = other DNA
gagggtctcgattacaatctgactcctgatgagg

SEQ ID NO: 21 - DNA, 35 bp, synthetic construct
  source 1..35: mol_type = other DNA
gagggtctcggatggtgcataaggctgaatacttg

SEQ ID NO: 22 - DNA, 33 bp, synthetic construct
  source 1..33: mol_type = other DNA
gagggtctcgattattcagataaccactccagg

SEQ ID NO: 23 - DNA, 35 bp, synthetic construct
  source 1..35: mol_type = other DNA
gagggtctcggatgccaaaagccattcttctgaag

SEQ ID NO: 24 - DNA, 33 bp, synthetic construct
  source 1..33: mol_type = other DNA
gagggtctcgattagtgcactttttgtatagac

SEQ ID NO: 25 - DNA, 35 bp, synthetic construct
  source 1..35: mol_type = other DNA
gagggtctcggatgagtagatttccagaactgaag

SEQ ID NO: 26 - DNA, 29 bp, synthetic construct
  source 1..29: mol_type = other DNA
gagggtctcgattattgagatccaatgcg

SEQ ID NO: 27 - DNA, 32 bp, synthetic construct
  source 1..32: mol_type = other DNA
gagggtctcggatggccatcgactctgatatc

SEQ ID NO: 28 - DNA, 31 bp, synthetic construct
  source 1..31: mol_type = other DNA
gagggtctcgattatacccattcaataggat

SEQ ID NO: 29 - DNA, 34 bp, synthetic construct
  source 1..34: mol_type = other DNA
gagggtctcggatgctgaaaagtcttgcaccaaa

SEQ ID NO: 30 - DNA, 32 bp, synthetic construct
  source 1..32: mol_type = other DNA
gagggtctcgattaaatgccactgagggtagc

SEQ ID NO: 31 - DNA, 34 bp, synthetic construct
  source 1..34: mol_type = other DNA
caatcgctagcatccaacatccaaagacgaaagg

SEQ ID NO: 32 - DNA, 42 bp, synthetic construct
  source 1..42: mol_type = other DNA
ggatagcatgcaccttatcaagatagctagaaatagaaatgg

SEQ ID NO: 33 - DNA, 37 bp, synthetic construct
  source 1..37: mol_type = other DNA
caatagcatgcaacatccaaagacgaaaggttgaatg

SEQ ID NO: 34 - DNA, 43 bp, synthetic construct
  source 1..43: mol_type = other DNA
catggtaccggtaccttatcaagatagctagaaatagaaatgg

SEQ ID NO: 35 - DNA, 34 bp, synthetic construct; identical to SEQ ID NO: 31
  source 1..34: mol_type = other DNA

SEQ ID NO: 36 - DNA, 43 bp, synthetic construct
  source 1..43: mol_type = other DNA
gatattgctcgagaccttatcaagatagctagaaatagaaatg

SEQ ID NO: 37 - DNA, 32 bp, synthetic construct
  source 1..32: mol_type = other DNA
caatctcgagaacatccaaagacgaaaggttg

SEQ ID NO: 38 - DNA, 44 bp, synthetic construct
  source 1..44: mol_type = other DNA
caaccatttctatttctagctatcttgataaggtcttaagtcca

SEQ ID NO: 39 - DNA, 43 bp, synthetic construct; identical to SEQ ID NO: 34
  source 1..43: mol_type = other DNA

SEQ ID NO: 40 - DNA, 34 bp, synthetic construct
  source 1..34: mol_type = other DNA
ttacttaagtccaacatccaaagacgaaaggttg

SEQ ID NO: 41 - DNA, 22 bp, synthetic construct
  source 1..22: mol_type = other DNA
tcacagacgcgttgaattgtcc

SEQ ID NO: 42 - DNA, 39 bp, synthetic construct
  source 1..39: mol_type = other DNA
ttgctcctcagcttagaagaactcgtccaacatcaagtg

SEQ ID NO: 43 - DNA, 24 bp, synthetic construct
  source 1..24: mol_type = other DNA
ttagtcttgctcctcagcttagcc

SEQ ID NO: 44 - DNA, 22 bp, synthetic construct
  source 1..22: mol_type = other DNA
tcacagacgcgttgaattgtcc

SEQ ID NO: 45 - DNA, 37 bp, synthetic construct
  source 1..37: mol_type = other DNA
ttttgcggccgcatgagcaatctacccccaacttttg

SEQ ID NO: 46 - DNA, 36 bp, synthetic construct
  source 1..36: mol_type = other DNA
aaaagcggccgcctagacaccaccatctagtcggtt

SEQ ID NO: 47 - DNA, 45 bp, synthetic construct
  source 1..45: mol_type = other DNA
actagatggtggtgtctagtcaagaggatgtcagaatgccatttg

SEQ ID NO: 48 - DNA, 46 bp, synthetic construct
  source 1..46: mol_type = other DNA
tctgacatcctcttgactagacaccaccatctagtcggttttctag

SEQ ID NO: 49 - DNA, 27 bp, synthetic construct
  source 1..27: mol_type = other DNA
aaacgctgtcttggaacctaatatgac

SEQ ID NO: 50 - DNA, 24 bp, synthetic construct
  source 1..24: mol_type = other DNA
aaactgtcagttttgggccatttg

SEQ ID NO: 51 - DNA, 25 bp, synthetic construct
  source 1..25: mol_type = other DNA
acgctgtcttggaacctaatatgac

SEQ ID NO: 52 - DNA, 31 bp, synthetic construct
  source 1..31: mol_type = other DNA
tacccattcaataggattttgtagtacctgc

SEQ ID NO: 53 - DNA, 20 bp, synthetic construct
  source 1..20: mol_type = other DNA
gagcttcttctacggccccc

SEQ ID NO: 54 - DNA, 27 bp, synthetic construct
  source 1..27: mol_type = other DNA
tccagcagagtaaaatttcctagggac

SEQ ID NO: 55 - DNA, 34 bp, synthetic construct
  source 1..34: mol_type = other DNA
ctcttttaggttttaagttgtgggaacagtaaca

SEQ ID NO: 56 - DNA, 20 bp, synthetic construct
  source 1..20: mol_type = other DNA
gtgggtgcttctttgcgtgg

SEQ ID NO: 57 - DNA, 27 bp, synthetic construct
  source 1..27: mol_type = other DNA
agaattgccatcaagagactcaggact

SEQ ID NO: 58 - DNA, 32 bp, synthetic construct
  source 1..32: mol_type = other DNA
gatagagagaaatcgcaaactttgagaggaag

(19) United States
(12) Patent Application Publication
(10) Pub. No.: US 2025/0051786 A1
(43) Pub. Date: Feb. 13, 2025

(54) EXPRESSION CONSTRUCTS AND METHODS OF GENETICALLY ENGINEERING METHYLOTROPHIC YEAST

(71) Applicant: Impossible Foods Inc., Redwood City, CA (US)

(72) Inventors: Smita Shankar, Millbrae, CA (US); Martin Andrew Hoyt, San Francisco, CA (US)

(21) Appl. No.: 18/786,876

(22) Filed: Jul. 29, 2024

Related U.S. Application Data
(63) Continuation of application No. 17/584,028, filed on Jan. 25, 2022, now Pat. No. 12,084,667.
Application No. 17/584,028 is a continuation of application No. 16/123,121, filed on Sep. 6, 2018, now Pat. No. 11,319,544.
Application No. 16/123,121 is a continuation of application No. 15/678,342, filed on Aug. 16, 2017, now Pat. No. 10,273,492.
Application No. 15/678,342 is a continuation of application No. 15/678,891, filed on Aug. 16, 2017, now Pat. No. 9,938,327.
Application No. 15/678,891 is a continuation of application No. PCT/US2016/031797, filed on May 11, 2016.
(60) Provisional application No. 62/313,491, filed on Mar. 25, 2016.
(60) Provisional application No. 62/236,506, filed on Oct. 2, 2015.
(60) Provisional application No. 62/222,388, filed on Sep. 23, 2015.
(60) Provisional application No. 62/220,366, filed on Sep. 18, 2015.
(60) Provisional application No. 62/203,052, filed on Aug. 10, 2015.
(60) Provisional application No. 62/185,921, filed on Jun. 29, 2015.
(60) Provisional application No. 62/183,074, filed on Jun. 22, 2015.
(60) Provisional application No. 62/159,899, filed on May 11, 2015.

(51) Int. Cl. C12N 15/81; C07K 14/39; C07K 14/415; C07K 14/805; C12N 1/16; C12N 15/63

(52) U.S. Cl. CPC C12N 15/815; C07K 14/39; C07K 14/415; C07K 14/805; C12N 1/16; C12N 15/635; C12N 15/81

(57) ABSTRACT

Methods and materials for genetically engineering methylotrophic yeast are provided.

### CLAIM OF PRIORITY

This application is a Continuation of U.S. patent application Ser. No. 17/584,028, filed Jan. 25, 2022, which is a Continuation of U.S. patent application Ser. No. 16/123,121, filed Sep. 26, 2018, issued as U.S. Pat. No. 11,319,544, which is a Continuation of U.S. patent application Ser. No. 15/678,342, filed Aug. 16, 2017, issued as U.S. Pat. No. 10,273,492, which is a Continuation of U.S. patent application Ser. No. 15/678,891, filed Aug. 16, 2017, issued as U.S. Pat. No. 9,938,327, which is a Continuation of PCT/US2016/031797, filed May 11, 2016, which claims the benefit of priority to U.S. Provisional Application No. 62/313,491, filed Mar. 25, 2016, U.S. Provisional Application No. 62/236,506, filed Oct. 2, 2015, U.S. Provisional Application No. 62/222,388, filed Sep. 23, 2015, U.S. Provisional Application No. 62/220,366, filed Sep. 18, 2015, U.S. Provisional Application No. 62/203,052, filed Aug. 10, 2015, U.S. Provisional Application No. 62/185,921, filed Jun. 29, 2015, U.S. Provisional Application No. 62/183,074, filed Jun. 22, 2015, and U.S. Provisional Application No. 62/159,899 filed May 11, 2015, the entire contents of which are hereby incorporated by reference.

### SEQUENCE LISTING

This application contains a Sequence Listing that has been submitted electronically as an XML text file named “38767-0048006_SL_ST26.XML”. The XML file, created on Mar. 11, 2024 is 70,188 bytes in size. The material in the XML file is hereby incorporated by reference in its entirety.

### TECHNICAL FIELD

This disclosure generally relates to DNA constructs and methods of using such DNA constructs to genetically engineer methylotrophic yeast.

### BACKGROUND

Methylotrophic yeast such as Pichia pastoris are commonly used for expression of recombinant proteins. Constructs that can be used to efficiently express one or more polypeptides in a methylotrophic yeast are provided herein.

### SUMMARY

This disclosure describes the use of P. pastoris strains that overexpress the transcriptional activator, Mxr1, from the AOX1 promoter to increase expression of transgenes that also are expressed from the AOX1 promoter, which significantly improves the recombinant production of one or more proteins. In addition, expression of Mxr1 from the AOX1 promoter creates a positive feedback loop that allows for expression of other transgenes from the AOX1 promoter in the absence of methanol, the normally obligate inducer, when repressing carbon sources are depleted. Expression of Mxr1 results in a significant increase in the amount of protein produced.

In one aspect, a methylotrophic yeast cell is provided that includes a recombinant nucleic acid molecule. The recombinant nucleic acid molecule typically includes an exogenous nucleic acid encoding a transcriptional activator operably linked to at least one methanol-inducible promoter element. Representative methylotrophic yeast can be of the genus Candida, Hansemila, Pichia or Toruplosis. A representative methylotrophic yeast is Pichia pastoris.

In some embodiments, the recombinant nucleic acid molecule is stably integrated into the genome of the methylotrophic yeast cell. In some embodiments, the recombinant nucleic acid molecule is extrachromosomally expressed from a replication-competent plasmid.

In some embodiments, the exogenous nucleic acid encoding a transcriptional activator comprises a Mxr1 sequence from Pichia pastoris, a Adr1 sequence from Hansenula polymorpha, a Trml sequence from Candida boidinii, and a Trm2 sequence from Candida boidinii. A representative nucleic acid encoding a transcriptional activator is shown in DQ395124. A representative transcriptional activator has an amino acid sequence shown in ABD57365.

In some embodiments, the at least one methanol-inducible promoter element is an alcohol oxidase 1 (AOX1) promoter element from Pichia pastoris, an AOD1 promoter element from Candida boidinii, a MOX promoter element from Hansenula polymorpha, a MOD1 promoter element from Pichia methanolica, a DHAS promoter element from Pichia pastoris, a FLD1 promoter element from Pichia pastoris, or a PEX8 promoter element from Pichia pastoris.

In some embodiments, the methylotrophic yeast cell further includes a nucleic acid molecule that includes at least one heterologous nucleic acid encoding a polypeptide operably linked to at least one methanol-inducible promoter element. In some embodiments, the at least one heterologous nucleic acid encodes one or more polypeptides involved in the biosynthesis of an iron co-factor such as heme (e.g., ALA synthase, ALA dehydratase, porphogilinogen deaminase, UPG III synthase, UPG III decarboxylase, CPG oxidase, PPG oxidase, and/or ferrochelatase). In some embodiments, one or more of the polypeptides involved in the biosynthesis of the iron co-factor are linked to at least one methanol-inducible promoter element.

In another aspect, a method for expressing a heterologous polypeptide in a cell is provided. Such a method typically includes providing a methylotrophic yeast cell as described herein; introducing a recombinant nucleic acid molecule into methylotrophic yeast cell, the recombinant nucleic acid molecule comprising at least one heterologous nucleic acid encoding a polypeptide operably linked to at least one Pichia pastoris alcohol oxidase 1 (AOX1) promoter element; and culturing the cell under conditions suitable for expression of the recombinant nucleic acid molecule, thereby expressing the heterologous polypeptide.

In some embodiments, the conditions under which the cells are cultured includes the addition of iron or a pharmaceutically or metabolically acceptable salt thereof. In some embodiments, the introducing step includes a technique such as transduction, electroporation, biolistic particle delivery, or chemical transformation. In some embodiments, the culturing step includes culturing the cell in the present of methanol.

In another aspect, a recombinant organism is provided that includes a transcriptional activator operably linked to the promoter it activates. In some embodiments, a recombinant organism is provided that expresses a polypeptide operably linked to the promoter. In yet another aspect, a method of expressing a polypeptide from an inducible promoter without addition of an inducer is provided as described herein.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic depicting the steps involved in the heme biosynthesis pathway.

FIG. 2A-2D are schematics of plasmids used in the construction of production strain MXY0183.

FIG. 3 is a schematic showing the generation of the production strains, MXY0183 and MXY0207, from the parent strain, Bg11.

FIG. 4A-4B are schematics showing plasmids pGAB and pMx354.

FIG. 5 is a schematic showing the generation of the antibiotic selection free production strains, MXY0291 and MXY0338, from the parent strain, Bg11.

FIG. 6 is a schematic of the linear pieces of DNA containing Mxr1 and LegH var 3 that were introduced by co-transformation to make the production strain MXY0291.

FIG. 7 is a schematic showing the linear construct expressing LegH under control of native Pichia non-pAOX1 constitutive promoters.

FIG. 8 is a photograph showing the phenotypic changes associated with strain, MXY0183. Shake flasks at the start of induction (0 hr) and 72 hr post-induction are shown. 1, MXY0051; 2, MXY0118; 3, MXY0183.

FIG. 9 shows the production of LegH from the modified P. pastoris strains. Panel A is a SDS gel showing lysates from P. pastoris strains grown in shake flasks: 51, MXY0051; 118, MXY0118; 183, MXY0183. Panel B is a table comparing LegH production from strains MXY0118, MXY0183, and MXY0207.

FIG. 10 shows data from experiments with strain MXY0206. Panel A is a photograph of shake flask cultures of strains MXY0183 (left) and MXY0206 (right) after 48 hr of growth in repressing carbon source. Panel B is a photograph of cell pellets from shake flask cultures of strains MXY0183 (left) and MXY0206 (right) after 48 hr of growth in BMY media. Panel C is a graph showing the relative yield of heme-loaded LegH (in the absence of any induction agent).

FIG. 11 is a summary table showing relative yields of strains described herein when grown in the presence of methanol with glycerol or methanol with glucose in 2 L fermenter tanks.

### DETAILED DESCRIPTION

Nucleic acid constructs are provided herein that allow for genetically engineering a cell to increase the recombinant expression of a polypeptide. In some embodiments, nucleic acid constructs are provided herein that allow for genetically engineering a cell to increase the recombinant expression of a polypeptide from an inducible promoter in the absence of the inducing molecule. Without being bound by any particular mechanism, the methods described herein create a positive feedback loop where the low level native expression of a transcriptional activator induces a promoter that is operably linked to a transcriptional activator. This leads to an increased expression of the transcriptional activator as well as one or more target polypeptides that are operably linked to the same inducible promoter.

Nucleic acid constructs are provided herein that allow for genetically engineering a methylotrophic yeast cell. While the methods are exemplified herein using a Pichia species (i.e., P. pastoris), other species of the Pichia genus can be used or species from any of the Candida, Hansenula, Pichia and Torulopsis genera.

Genetically engineering a methylotrophic yeast cell typically includes introducing a recombinant nucleic acid molecule into the cell. As described herein, a recombinant nucleic acid molecule typically includes an exogenous nucleic acid that encodes a transcriptional activator operably linked to at least one inducible promoter element.

Recombinant nucleic acid molecules used in the methods described herein are typically DNA, but RNA molecules can be used under the appropriate circumstances. As used herein, “exogenous” refers to any nucleic acid sequence that is introduced into the genome of a cell from an external source, where the external source can be the same or a different organism or a nucleic acid generated synthetically. For example, an exogenous nucleic acid can be a nucleic acid from one microorganism (e.g., one genus or species of methylotrophic yeast) that is introduced into a different genus or species of methylotrophic yeast, however, an exogenous nucleic acid also can be a nucleic acid from a methylotrophic yeast that is introduced recombinantly into a methylotrophic yeast as an additional copy despite the presence of a corresponding native nucleic acid sequence. For example, P. pastoris contains an endogenous nucleic acid encoding a Mxr1transcriptional activator; an additional P. pastoris Mxr1 nucleic acid (e.g., introduced recombinantly into P. pastoris) or modifying the endogenous P. pastoris Mxr1 nucleic acid is considered exogenous.

Transcriptional activators, and nucleic acids encoding transcriptional activators (e.g., exogenous nucleic acids encoding transcriptional activators), are known in the art. For example, a transcriptional activator from Pichia pastoris is the Mxr1 sequence, but suitable transcriptional activators also can be found in Hansenula polymorpha (the Adr1 sequence; see, for example, GenBank Accession No. AEOI02000005, bases 858873 to 862352, for the nucleic acid sequence and GenBank Accession No. ESX01253 for the amino acid sequence) and Candida boidinii (the Trm1 sequence; see, for example, GenBank Accession No. AB365355 for the nucleic acid sequence and GenBank Accession No. BAF99700 for the amino acid sequence; the Trm2 sequence; see, for example, GenBank Accession No. AB548760 for the nucleic acid sequence and GenBank Accession No. BAJ07608 for the amino acid sequence). A representative P. pastoris Mxr1 nucleic acid sequence can be found, for example, in GenBank Accession No. DQ395124, while a representative P. pastoris Mxr1 polypeptide sequence can be found, for example, in GenBank Accession No. ABD57365.

Transcriptional activators such as Mxr1 may be normally expressed at low levels. Therefore, it is desirable to place the exogenous nucleic acid (i.e., the transcriptional activator) under control of a promoter that is inducible. As used herein, “operably linked” means that a promoter or other expression element(s) are positioned relative to a nucleic acid coding sequence in such a way as to direct or regulate expression of the nucleic acid (e.g., in-frame).

There are a number of inducible promoters that can be used when genetically engineering methylotrophic yeast. For example, a methanol-inducible promoter, or a promoter element therefrom, can be used. Methanol inducible promoters are known in the art. For example, a commonly used methanol-inducible promoter from P. pastoris is the promoter, or a portion thereof, from the alcohol oxidase 1 (AOX1) gene, which is strongly transcribed in response to methanol. Other methanol-inducible promoters, or promoter elements therefrom, however, can be used, including, without limitation, the alcohol oxidase (AOD1) promoter from Candida boidinii (see, for example, GenBank Accession No. YSAAOD1A), the alcohol oxidase (MOX) promoter from Hansenula polymorpha (see, for example, GenBank Accession No. X02425), the MOD1 or MOD2 promoter from Pichia methanolica (see, for example, Raymond et al., 1998, Yeast, 14:11-23; and Nakagawa et al., 1999, Yeast, 15:1223-30), the DHAS promoter from P. pastoris (see, for example, GenBank Accession No. FJ752551) or a promoter element therefrom, the formaldehyde dehydrogenase (FLD1) promoter from Pichia pastoris (see, for example, GenBank Accession No. AF066054), or the PEX8 promoter from P. pastoris (see, for example, Kranthi et al., 2010, Yeast, 27:705-11). In some embodiments, the transcriptional activator is a Mitl sequence from Pichia pastoris (see, for example, GenBank Accession No. CAY70887). All of these promoters are known to be induced by methanol.

A skilled artisan would understand that the recombinant nucleic acid molecule described herein can be stably integrated into the genome of the methylotrophic yeast cell, or can be extrachromosomally expressed from a replication-competent plasmid. Methods of achieving both are well known and routinely used in the art.

As demonstrated herein, the methanol-regulated transcriptional activators in Pichia can bind to the AOX1 promoter and act cooperatively with Mxr1 to activate transcription from the AOX1 promoter. In some embodiments, two methanol-regulated transcriptional activators (e.g., Mxr1 and Mit1) can be operably linked to a methanol inducible promoter element.

A strain that includes a recombinant nucleic acid molecule as described herein can be used to regulate (e.g., overexpress) a second recombinant nucleic acid molecule in the methylotrophic yeast cell. A second recombinant nucleic acid molecule can include, for example, one or more heterologous nucleic acids encoding one or more polypeptides of interest. Similar to the exogenous nucleic acid encoding the transcriptional activator, a heterologous nucleic acid refers to any nucleic acid sequence that is not native to the genome or in the genome of an organism (e.g., a heterologous nucleic acid can be a nucleic acid from one microorganism (e.g., one genus or species of methylotrophic yeast) that is introduced into a different genus or species of methylotrophic yeast).

Simply by way of example, heterologous nucleic acids encoding the one or more polypeptides of interest can be the nucleic acids involved in the biosynthesis of a heme-co-factor. Exemplified herein are nucleic acids encoding the 8 different enzymes involved in heme biosynthesis as determined and annotated from the sequence of the Pichia pastoris genome. For example, heterologous nucleic acids encoding ALA synthase, ALA dehydratase, porphobilinogen deaminase, UPG III synthase, UPG III decarboxylase, CPG oxidase, PPG oxidase, and ferrochelatase can be expressed in the methylotrophic yeast strains described herein. For genetically engineering methylotrophic yeast to contain more than one heterologous nucleic acids (e.g., transgenes), a combination of methanol-inducible and constitutive promoters, or elements therefrom, can be combined to further increase the expression of such nucleic acids.

Previous studies in Saccharomyces cerevisiae identified ALA dehydratase and porphobilinogen deaminase as rate limiting enzymes in heme biosynthesis (see, for example, Hoffman et al., 2003, Biochem. Biophys. Res. Commun., 310(4):1247-53). However, heterologous expression of individual heme enzymes in P. pastoris from the glyceraldehyde-3-phosphate dehydrogenase (GAP) promoter failed to overcome limitations associated with the expression of a recombinant protein containing a heme co-factor (see Krainer et al., 2015, Microb. Cell Fact., 13;14:4). As described herein, highly efficient expression of a recombinant heme containing protein in P. pastoris was achieved by co-expressing the entire heme biosynthetic pathway from methanol-inducible promoters, although it would be appreciated that one or more of the genes involved in the heme biosynthetic pathway could be expressed from one or more constitutive promoters.

In addition to the enzymes involved in iron-co-factor biosynthesis, it would be understood that a nucleic acid encoding a member of the globin family of proteins (PF00042 in the Pfam database) including plant hemoglobins can be present. In the Examples herein, a nucleic acid encoding soybean leghemoglobin (LegH) is present. LegH is a protein that binds to the iron co-factor, heme, which results in a characteristic absorption at 415 nm and a distinct red color. The LegH protein (also known as LGB2) is naturally found in root nodules of soybean (see, for example, UniprotKB Accession No. P02236), and the nucleic acid sequence used herein was codon optimized for expression in P. pastoris. See, for example, WO 2014/110539 and WO 2014/110532.

Alternatively, a heterologous nucleic acid encoding a polypeptide of interest can be, for example and without limitation, a dehydrin, a phytase, a protease, a catalase, a lipase, a peroxidase, an amylase, a transglutaminase, an oxidoreductase, a transferase, a hydrolase, a lyase, an isomerase, a ligase, or an antibody against any such polypeptides. In other embodiments, a heterologous nucleic acid can encode one or more enzymes involved in the pathway for production of small molecules, such as ethanol, lactic acid, butanol, adipic acid, or succinic acid.

Similar to the exogenous nucleic acid encoding the transcriptional activator, the heterologous nucleic acid encoding a polypeptide of interest can be operably linked to an inducible promoter element (e.g., a methanol-inducible promoter element), or the heterologous nucleic acid encoding a polypeptide of interest can be operably linked to a constitutive promoter or constitutive promoter element. Inducible promoters and elements therefrom are discussed above. Constitutive promoters and constitutive promoter elements are known in the art. For example, a commonly used constitutive promoter from P. pastoris is the promoter, or a portion thereof, from the transcriptional elongation factor EF-1α gene (TEF1), which is strongly transcribed in a constitutive manner. Other constitutive promoters, or promoter elements therefrom, however, can be used, including, without limitation, the glyceraldehyde-3-phosphate dehydrogenase (GAPDH) promoter from P. pastoris (see, for example, GenBank Accession No. U62648.1), the promoter from the potential glycosyl phosphatidyl inositol (GPI)-anchored protein, GCW14p (PAS_chr1-4_0586), from P. pastoris (see, for example, GenBank Accession No. XM_002490678), or the promoter from the 3-phosphoglycerate kinase gene (PGK1) from P. pastoris (see, for example, GenBank Accession No. AY288296).

Similar to the recombinant nucleic acid molecule described herein, the second recombinant nucleic acid molecule can be stably integrated into the genome of the methylotrophic yeast cell, or can be extrachromosomally expressed from a replication-competent plasmid.

It would be understood by the skilled artisan that a combination of inducible (e.g., methanol-inducible) and constitutive promoters (or promoter elements therefrom) can be combined to further increase the expression of any of the nucleic acids operably linked thereto.

It would be appreciated by a skilled artisan that a heterologous nucleic acid encoding a polypeptide of interest operably linked to a promoter element can be separate from the recombinant nucleic acid molecule described herein, or can be contiguous with the exogenous nucleic acid encoding a transcriptional activator operably linked to a promoter element contained within the recombinant nucleic acid molecule described herein. It also would be appreciated by a skilled artisan that, if the second nucleic acid molecule is contiguous with the recombinant nucleic acid molecule described herein, that a single promoter, or promoter element therefrom, can be used to drive transcription of both or all of the genes (e.g., the exogenous nucleic acid encoding the transcriptional activator as well as the one or more heterologous nucleic acids encoding the polypeptide(s) of interest).

Methods of introducing nucleic acids into methylotrophic yeast cells are known in the art, and include, without limitation, transduction, electroporation, biolistic particle delivery, and chemical transformation.

In addition, methods of culturing methylotrophic yeast cells are known in the art. See, for example, Pichia Protocols, Methods In Molecular Biology, 389, Cregg, Ed., 2007, 2^{nd} Ed., Humana Press, Inc. Under some circumstances, it may be desirable to introduce or add methanol to the culture media, although, as demonstrated herein, methanol is not required to obtain efficient expression at high levels of one or more polypeptides of interest. Under some circumstances (e.g., when one or more nucleic acids encoding enzyme(s) involved in an iron-co-factor biosynthesis are expressed), it may be desirable to supplement the culture media with iron or a pharmaceutically or metabolically acceptable (or GRAS) salt thereof.

Pichia strains are able to grow on methanol as the sole carbon source. Methanol utilization is initiated by the conversion of methanol to formaldehyde by the action of alcohol oxidase. The methylotrophic yeast, Pichia pastoris, contains two genes for alcohol oxidases, AOX1 and AOX2. Strains with reduced alcohol oxidase activity (“methanol utilization slow” or MutS strains) often produce more of a recombinant protein expressed from the AOX1 promoter than strains that do not have reduced alcohol oxidase activity. Strains mutated in both AOX genes and completely lacking alcohol oxidase activity cannot metabolize methanol, but can still be induced for expression from the AOX1 promoter by methanol. These strains retain the ability to use other carbon sources for growth, but still express heterologous proteins from the AOX1 promoter upon the addition of methanol. Because these strains do not metabolize methanol (“methanol utilization minus” or Mut-strains), much less methanol is required for induction of protein expression, and strains carrying these mutations avoid issues related to methanol feeding in large-scale fermentations. See, for example, Chiruvolu et al., 1997, Enzyme Microb. Technol., 21:277-83. It was determined herein that expression of LegH from the AOX1 promoter in Mut-strains greatly improved the LegH yield. Thus, a methylotrophic yeast having a mutation in both the AOX1 gene and the AOX2 gene can be used in the methods described herein.

The protein of interest, or a complex that includes one or more proteins of interest (e.g., heme-bound LegH, a dehydrin, a phytase, a protease a catalase, a lipase, a peroxidase, an amylase, a transglutaminase, an oxidoreductase, a transferase, a hydrolase, a lyase, an isomerase, a ligase, or an antibody) can be purified from the yeast cells. Methods of purifying polypeptides are known in the art. As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the polypeptides and naturally occurring molecules with which it is naturally associated. Since a polypeptide that is chemically synthesized is, by nature, separated from the components that naturally accompany it, a synthetic polypeptide is “purified.”

As used herein, nucleic acids can include DNA and RNA, and includes nucleic acids that contain one or more nucleotide analogs or backbone modifications. A nucleic acid can be single stranded or double stranded, which usually depends upon its intended use. Also provided are nucleic acids and polypeptides that differ from a given sequence. Nucleic acids and polypeptides can have at least 50% sequence identity (e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a given nucleic acid or polypeptide sequence.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31 (13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

Changes can be introduced into a nucleic acid molecule, thereby leading to changes in the amino acid sequence of the encoded polypeptide. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5 (Suppl. 3):345-352), which provides frequency tables for amino acid substitutions), and a non-conservative substitution is one in which an amino acid residue is replaced with an amino acid residue that does not have a similar side chain. Nucleic acid and/or polypeptide sequences may be modified as described herein to improve one or more properties including, without limitation, increased expression (e.g., transcription and/or translation), tighter regulation, deregulation, loss of catabolite repression, modified specificity, secretion, thermostability, solvent stability, oxidative stability, protease resistance, catalytic activity, and/or color.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A construct or vector containing a nucleic acid (e.g., a nucleic acid that encodes a polypeptide) also is provided. Constructs or vectors, including expression constructs or vectors, are commercially available or can be produced by recombinant DNA techniques routine in the art. A construct or vector containing a nucleic acid can have expression elements operably linked to such a nucleic acid, and further can include sequences such as those encoding a selectable marker (e.g., an antibiotic resistance gene). A construct or vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6xHis tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins.

Vectors as described herein can be introduced into a host cell. As used herein, “host cell” refers to the particular cell into which the nucleic acid is introduced and also includes the progeny of such a cell that carry the vector. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2× SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, CA).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

Methods are described herein that can be used to generate a strain that lacks sequences for selection (i.e., that lacks a selectable marker). These methods include using a circular plasmid DNA vector and a linear DNA sequence; the circular plasmid DNA vector contains a selection marker and an origin of DNA replication (also known as an autonomously replicating sequence (ARS)), and the linear DNA sequence contains sequences for integration into the Pichia genome by homologous recombination. The linear DNA molecule additionally can include nucleic acid sequences encoding one or more proteins of interest such as, without limitation, heme-bound LegH, a dehydrin, a phytase, a protease a catalase, a lipase, a peroxidase, an amylase, a transglutaminase, an oxidoreductase, a transferase, a hydrolase, a lyase, an isomerase, a ligase, one or more enzymes involved in the pathway for production of small molecules, such as ethanol, lactic acid, butanol, adipic acid or succinic acid, or an antibody against any such proteins.

Pichia cells can be transformed with both DNA molecules and the transformants selected by the presence of the selectable marker on the circular plasmid. Transformants then can be screened for integration of the linear DNA molecule into the genome using, for example, PCR. Once transformants with the correct integration of the marker-free linear DNA molecule are identified, the cells can be grown in the absence of selection for the circular plasmid. Because the marker-bearing plasmid is not stably maintained in the absence of selection, the plasmid is lost, often very quickly, after selection is relaxed. The resulting strain carries the integrated linear DNA in the absence of heterologous sequences for selection. Therefore, this approach can be used to construct Pichia strains that lack a selectable marker (e.g., a heterologous selection marker) with little to no impact on recombinant protein yield.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### PART A. Materials and Methods

### Example 1—Polymerase Chain Reaction

Genes of interest were amplified from genomic DNA or plasmid DNA templates using PHUSION® Hi-fidelity DNA polymerase (New England Biolabs). Briefly, 0.6 μM each of forward and reverse primers are incubated with 10-50 ng of template DNA and 400 μM of nucleotide mix in the presence of 1-2 U of PHUSION® DNA polymerase. The reaction conditions were as follows in Table 1:

TABLE 1

| | | | |
|---|---|---|---|
| 1 cycle | Initial Denaturation | 98° C. | 1 min |
| 25 cycles | Denaturation | 98° C. | 10 sec |
| | Annealing | | 20 sec |
| | Extension | 72° C. | 30 sec per kb |
| 1 cycle | Final Extension | 72° C. | 5 min |
| 1 cycle | Hold | 4° C. | Forever |

### Example 2—Plasmid Construction by Ligation

50-100 ng of restriction enzyme digested plasmid and 3X molar excess of PCR amplified inserts were incubated in the presence of T4 DNA ligase (New England Biolabs). Ligation was carried out at 16° C. for greater than 2 hr. 2 μl of ligation reaction was transformed into DH10B electrocompetent E. coli cells

Example 3—Transformation into E. Coli ElectroMax DH10B T1 Phage-Resistant Competent Cells

1.5-2 ul of ligation mixture was transformed into 20 ul of ElectroMax DH10B T1 Phage-Resistant Competent Cells (Invitrogen, Cat #12033-015) by electroporation using MICROPULSER™(BioRad) set at 1.7 kV using a 1 mm gap cuvette (BioRad, Cat #165-2089); after a pulse 1 ml SOC was added to cells and cells were incubated at 37° C. for 1 h with shaking at 200 rpm. 10 μl of recovery mixture was plated on LB agar plates containing ampicillin at a concentration of 100 μg/ml. Plates were incubated overnight at 37° C.

Example 4—Linearization of Plasmid DNA for Transformation into P. Pastoris

Plasmid DNA was digested with either Pmel restriction endonuclease (New England BioLabs, Cat #R0560L) in 1× CUTSMART® Buffer for 1-4 hours at 37° C. or Sfil restriction endonuclease in 1× CUTSMART® Buffer for 1-4 hours at 50° C. (New England BioLabs, Cat #R0123L). Linearized plasmid was gel purified from a 0.8% agarose gel using ZYMOCLEAN™ Gel DNA Recovery Kit (Zymo Research, Cat #D4002). DNA was eluted in 20 μl H₂O.

### Example 5—Preparation of P. Pastoris Transformation-Competent Cells

Selected strains of P. pastoris were grown to mid-exponential growth phase (˜2 OD) in 25 ml YPD medium. Cells were collected by centrifugation at 930 x g for 15 minutes. The cell pellet was resuspended in 2 ml of a solution of 80% YPD and 200 mM HEPES, pH 6.8. 75 μl of 1 M DTT was added. The resuspended cell pellet was mixed at 100 rpm at 30° C. for 25 minutes. A 40 ml volume of ice cold, sterile water was added to the suspension, and the cells were collected by centrifugation at 1125 x g for 15 minutes and placed on ice. The cell pellet was resuspended in 40 ml ice cold water and collected as before for two additional wash steps. The cell pellet was then resuspended in 20 ml of ice cold 1 M sorbitol and collected by centrifugation as before. The final cell pellet was suspended in 0.3 ml ice cold, sterile sorbitol, aliquoted and frozen at −80° C.

Example 6—Transformation into P. Pastoris

30-100 ng of linearized plasmid DNA was transformed into 30 μl of electrocompetent P. pastoris cells using a 1 mm gap GenePulser cuvette (BioRad) with a GenePulser (BioRad) set at 1.15 kV. 1 ml of YPD/1M sorbitol was added and mixed at a 1:1 ratio to the cells. The cells were allowed to recover for 3 h at 30° C. with shaking at 100 rpm. 100 μl of the recovery mixture was plated on a YPD plate containing the appropriate antibiotic, and the rest of the cells were plated on a YPD plate with the appropriate antibiotic. Plates were incubated at 30° C. for 48 hours. Primary transformation plates were streaked onto YPD plates with appropriate antibiotic, and plates were incubated for 48 h at 30° C. Individual clones were patched onto YPD plates with antibiotics and the patches were used to do colony PCR or gDNA prep to confirm integration into the chromosome and to grow the strains in shake flasks for further analysis.

### Example 7—Growing Cultures in Shake Flasks for Production of LegH

A strain from a fresh patch was inoculated into growth media BMGY (BMY supplemented with 0.75% glycerol) and grown overnight at 30° C. with shaking at 200 rpm. The next day, expression of LegH was induced with methanol by diluting the ON culture with BMMY media (BMY+1% methanol) supplemented with 0.1 mM Ammonium Fe (III) citrate. The culture was grown to an OD600 of 0.5-0.7. Antifoam was added to a final concentration of 0.01%. The cultures were grown for 72 hours total; cultures were supplemented with methanol every 24 hours by adding 1/10 of shake flask volume of 10× BMMY media (BMY+10% methanol). Cells were harvested after 72 h of induced growth by centrifugation.

### Example 8—Shake Flask Medium

BMY media (Table 2) was prepared by dissolving 10 g of yeast extract and 20 g soytone in 790 ml water. The mixture was sterilized by autoclaving, and cooled to room temperature. 100 ml 1 M Potassium Phosphate buffer (pH 6.0) and 100 ml 10X Yeast Nitrogen Base without amino acids (13.4 g of YNB powder per 100 mL; Sigma-Aldrich) was filter sterilized (0.2 μm pore size PES) and added to the media. No pH adjustment is required.

TABLE 2
BMY Media Components

| Component | Amount, per 1 L |
|---|---|
| Yeast Extract | 10 g |
| Soy peptone (BD) | 20 g |
| Yeast Nitrogen Base without amino | 100 mL (results in 13.4 |
| acids, 10X solution | g/L in BMY) |
| 1 M Potassium Phosphate Buffer, pH 6.0 | 100 mL |

The following components in Table 3 were dissolved in water, and autoclaved to sterilize.

TABLE 3
Low-Osmolarity Medium for Shake Flask

| Component | Amount, g/L |
|---|---|
| Ammonium Sulfate | 15.7 |
| Potassium Phosphate Monobasic | 9.4 |
| Calcium Sulfate Dihydrate | 0.43 |
| Magnesium Sulfate Heptahydrate | 11.7 |
| Sodium Citrate Dihydrate | 1.13 |

### Example 9—Fermentation Medium and Feeds

The components indicated below (Table 4) were dissolved and the volume adjusted with water. The components were FCC food grade or equivalent. The medium was sterilized by autoclaving, by steaming in place, or with an equivalent.

TABLE 4
Low-Osmolarity Medium with 95 g/L Glycerol for Fermentation

| Component | Amount, g/L |
|---|---|
| Ammonium Sulfate | 15.7 |
| Potassium Phosphate Monobasic | 9.4 |
| Calcium Sulfate Dihydrate | 0.43 |
| Magnesium Sulfate Heptahydrate | 11.7 |
| Sodium Citrate Dihydrate | 1.13 |
| Glycerol, USP grade 99.7% | 95 |

After sterilization, the medium was allowed to cool down to room temperature, and the following in Table 5 was added:

TABLE 5
Additional Components

| Component | Amount, mL/L |
|---|---|
| Trace Metals PTM1 Solution | 2 |
| Vitamin Solution | 4 |
| Sigma 204 antifoam or equivalent | 1 |

Trace metals PTM1 solution is available as a powdered mix from Sunrise Science (Cat No. 4052-A-B-1L) (Table 6). Pouch A and pouch B were mixed in 950 mL water, and 5 mL sulfuric acid was added. Some precipitation is expected upon mixing; the mixture was filter sterilized (0.2 μm pore size PES) and stored at 4° C. in the dark.

TABLE 6
Vitamin solution recipe

| Component | Amount, g/L |
|---|---|
| biotin | 0.2 |
| calcium pantothenate | 1 |
| folic acid | 0.2 |
| inositol | 1 |
| niacin | 0.2 |
| p-aminobenzoic acid | 0.2 |
| Pyridoxine hydrochloride | 1 |
| riboflavin | 0.5 |
| thiamine hydrochloride | 1 |
| B12 | 0.1 |

Alternatively, trace metals PTM1 can be made as follows (Table 7):

TABLE 7
Trace metal PTM1

| Component | Amount | |
|---|---|---|
| Cupric sulfate—5H2O | 6.0 | g |
| Sodium iodide | 0.08 | g |
| Manganese sulfate—H2O | 3.0 | g |
| Sodium molybdate—2H2O | 0.2 | g |
| Boric acid | 0.02 | g |
| Cobalt chloride | 0.5 | g |
| Zinc chloride | 0.5 | g |
| Ferrous sulfate—7H2O | 65.0 | g |
| Biotin | 0.2 | g |
| Sulfuric acid | 5.0 | ml |

| | |
|---|---|
| Water | To a final volume of 1 L |

The components are mixed together, filter sterilized and stored at room temperature. The glycerol feed mix was prepared by mixing 17.5 g of AmberFerm 4000 into 320 mL water and stirring to dissolve. The water-Amberferm mixture was added to 850 g of glycerol and mixed well by vigorous stirring. The feed mix was sterilized by autoclaving. See Table 8.

TABLE 8
Glycerol feed solution

| Component | Amount, g/L |
|---|---|
| USP grade glycerol | 850 |
| Water | 320 |
| Sensient AmberFerm4000 soy hydrolysate | 17.5 |

The methanol feed was made using 99-100% methanol supplemented with 12 mL/L of PTM1 solution.

### Example 10—Protocol for Lab-Scale High Oxygen Transfer Fermentation

### Seed Shake Flask Protocol

In a aseptic biosafety hood, low-osmolarity medium and BMY were mixed in a 9:1 low-osmo: BMY ratio. Glycerol, at a concentration of 12.5 g/L, was added to the medium. USP food grade glycerol/glycerin (99.7% purity in a 50% v/v (63% w/w) glycerol/water solution) was used and autoclaved to sterilize. Sigma 204 or an equivalent antifoam was added to the medium at a concentration of 0.25 mL/L. Glycerol seed vials were retrieved, sprayed outside with 70% IPA or ethanol and thawed inside a biosafety hood at room temperature for about 5 min. Baffled shake flasks were inoculated with glycerol seed vials; 1 mL of inoculum vial were used for every 1 L of shake flask medium. Cultures were grown at 30° C. for 24 hours with shaking (200 RPM with a 1″ throw). A ratio of between 1:10 and 1:5 of actual medium volume:nominal shake flask volume was used. 2.8 L nominal volume flask with 250 to 500 mL of medium were routinely use with success. The OD at 600 nm was measured after 24 hours of growth; if the OD was 15 or higher, the culture was used to inoculate a fermenter. If the OD was less than 15, the culture was grown for 1-2 more hours before the OD was determined again. If an OD of 15 was not reached after 15 to 30 hours, the seed flask was considered to have failed.

### Fermentation Protocol

The fermentation medium and feeds were prepared as described herein. The initial volume should be about 40% of the maximum fermenter volume, e.g., 4 L, if the maximum working volume of the fermenter is 10 L. This is because the process will approach the maximum working volume by the end of the fermentation. The fermenter is inoculated with shake flask seed at 10% inoculum-fermenter ratio, e.g. if 4 L of initial media are present in the fermenter, the fermenter is inoculated with about 0.4 L of shake flask seed. The total volume in the fermenter at this point is referred to T0 volume, e.g. 4.4 L in this representative example. Process controls include the following: 30° C. temperature; dissolved oxygen controlled by agitation-acration cascade to maintain a 20% saturation set point; and pH controlled via addition of 28% NH₄OH, the set point will depend on the phase of the process.

Batch phase (from inoculation to depletion of glycerol, signaled by DO spike): Depending on the responsiveness of the PID control for dissolved oxygen, a strong DO spike or a fast drop in agitation-aeration rates or a combination of both may be observed when the cells deplete the glycerol present in the medium. Fed-batch phase is initiated when this occurs. The duration of the batch phase is approximately 20 hours, but up to 24 hours is considered acceptable. The pH set point is 5.0. The wet cell weight at the end of the batch phase will be approximately 220 g/L.

Fed-batch phase: glycerol feed is initiated to achieve 12-14 g/L/hr of neat glycerol based on T0 volume. The federate was maintained until approximately 350 g/L wet cell weight was reached, which should take about 7-10 hours. The pH set point is 5.0.

Transition phase: A sample is taken before beginning the transition phase. Methanol feed was initiated to achieve 1 g/L/hr of neat methanol, based on the T0 volume, until 1-2 g/L methanol concentration was reached in the fermentation broth. The methanol feed rate was adjusted during the remainder of fermentation so as to maintain a methanol concentration of 0.25-1 g/L in broth. Glycerol federate was reduced from 12-14 g/L/hr to 8-9 g/L/hr of neat glycerol, based on T0 volume, linearly over the course of 2 hours. Stepwise reduction in feed rate every 20 min would be acceptable as well. The pH set point was changed to 3.5, and the fermentation was allowed to naturally adjust to the new set point (i.e., with no addition of acid).

Production phase (from end of glycerol feed ramp-down to end of fermentation): The pH set point was 3.5. A methanol concentration of 0.25-1 g/L was maintained in the fermentation broth. The feed rate of the glycerol was maintained at 8-9 g/L/hr of neat glycerol, based on the TO volume. Samples were taken approximately every 12 hours. Samples were spun at 4000 to 7000 RCF at 4° C., and the supernatant decanted. The supernatant was saved in a separate tube. Pellets and 3 samples of 5 mL of supernatants at each time point were frozen at −80° C. If a 15-20% DO during production is unable to be maintained, even at maximum aeration and agitation rates for the vessel, the glycerol feed rate can be lowered up to 5 g/L/hr of neat glycerol, based on the TO volume. Fermentation ended 60 hours after inoculation. At 1000 L scale, the harvest process consisted of shutting down feeds and acration, chilling the broth to 8° C., and concentrating the paste using a sharples or disk stack centrifuge. Harvesting usually takes about 5-10 hours and does not incur a detectable loss of quality of the product. For lab scale, it is sufficient to collect, in addition to the 3×5 mL samples, an additional 50 mL sample at the end. Wet cell weight was >450 g/L, and spun pellets looked pink, as opposed to spun pellets from pre-induction samples, which looked more white. The color change of the broth from white to a more pronounced pink started following about 6-12 hours of induction.

### PART B. Construction of Production Strains

### Production Strain MXY0183

Example 11—Cloning Each Enzyme of Heme Biosynthesis Pathway into pGAN or pGAZ Integration Vector

pGAN (with the nat selection marker) and pGAZ (with the ZEOCIN™ selection marker) were purchased from Biogrammatics, Inc (Carlsbad, CA). Each gene was placed under control of the AOX1 promoter, and the FDH terminator was placed immediately after the stop codon of each gene. The genes in the heme biosynthesis pathway were PCR amplified from wild type P. pastoris strain or subcloned from previous constructs.

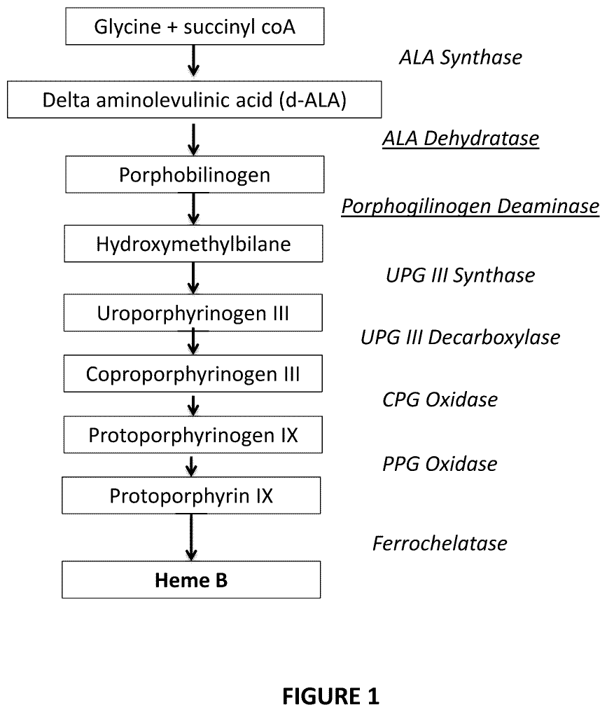

The heme biosynthetic pathway, including the enzymes involved in the biosynthesis of heme, are shown in FIG. 1. The intermediates produced during the biosynthesis of heme are shown in the boxes, and the enzyme catalyzing each step is shown to the right. The rate limiting enzymatic steps, as shown in S. cerevisiae, are shown with underlining.

ALA synthase, ALA dehydratase, UPGIII synthase, UPGIII decarboxylase, CPG oxidase and PPG oxidase genes were PCR amplified with primers containing sites for recognition by the restriction endonuclease, BsaI (Table 9). Oligonucleotides were synthesized by ElimBiopharm.

TABLE 9

| Primer | | | SEQ ID |
|---|---|---|---|
| Designation | Gene | Sequence | NO: |
| MxO0187 | ALAsynth_F | GAGGGTCTCGGATGGAGTTTGTCGCCCGTC | 19 |
| MxO0188 | ALAsynth_R | GAGGGTCTCGATTACAATCTGACTCCTGATGAGG | 20 |
| MxO0189 | ALAdehyd_F | GAGGGTCTCGGATGGTGCATAAGGCTGAATACTTG | 21 |
| MxO0190 | ALAdehyd_R | GAGGGTCTCGATTATTCAGATAACCACTCCAGG | 22 |
| MxO0191 | UroporSynth_F | GAGGGTCTCGGATGCCAAAAGCCATTCTTCTGAAG | 23 |
| MxO0192 | UroporSynth_R | GAGGGTCTCGATTAGTGCACTTTTTGTATAGAC | 24 |
| MxO0193 | UroporDecarb_F | GAGGGTCTCGGATGAGTAGATTTCCAGAACTGAAG | 25 |
| MxO0194 | UroporDecarb_R | GAGGGTCTCGATTATTGAGATCCAATGCG | 26 |
| MxO0195 | CoproOx_F | GAGGGTCTCGGATGGCCATCGACTCTGATATC | 27 |
| MxO0196 | CoproOx_R | GAGGGTCTCGATTATACCCATTCAATAGGAT | 28 |
| MxO0197 | ProtoporOx_F | GAGGGTCTCGGATGCTGAAAAGTCTTGCACCAAA | 29 |
| MxO0198 | ProtoporOx_R | GAGGGTCTCGATTAAATGCCACTGAGGGTAGC | 30 |

PHUSION® High-Fidelity DNA Polymerase (New England BioLabs, Cat #M0530L) was used to amplify genes from genomic DNA. PCR products were obtained and purified using DNA Clean&Concentrator-5 (Cat #D4004) and DNA was eluted in 25 μl of H₂O. Vector DNA, pGAZ and pGAN, and PCR products were digested with BsaI (New England BioLabs, Cat #R0535S) in 50 μl reaction volume at 37° C.

Linearized vectors and digested PCR products were purified from 0.8% agarose gel using ZYMOCLEAN™ Gel DNA Recovery Kit (Zymo Research Cat #D4002). DNA was eluted in 20 μl H₂O. Ligation reactions were set up in 10 μl at 16° C. overnight using T4 DNA Ligase (New England BioLabs, Cat #M0202S).

PBD and ferrochelatase genes were subcloned from previously constructed plasmids: pJAZ_PBD was digested with BstBI (Bsp1191) (ThermoScientific, FD0124) and NotI (ThermoScientific, FD0596) in 1× Fast Digest buffer for 5 min at 37° C. pJAZ_Ferroch was digested with Mfel (MunI, ThermoScientific, FD0753) and NotI (ThermoScientific, FD0596) in 1× Fast Digest buffer for 5 min at 37° C.

Digested products were purified from 0.8% agarose gel using ZYMOCLEAN™ Gel DNA Recovery Kit (Zymo Research Cat #D4002). DNA was eluted in 20 μl H₂O.

Ligation reactions were set up in 10 ul reaction at 16° C. overnight using T4 DNA Ligase (New England BioLabs, Cat #M0202S).

1.5 ul of ligation mixture was transformed into 20 ul of ElectroMax DH10B T1 Phage-Resistant Competent Cells (Invitrogen, Cat #12033-015) by electroporation using MICROPULSER™ (BioRad) set at 1.7 kV; cells were incubated at 37° C. in 1 ml SOC for 1 h with shaking at 200 rpm. 10 μl of recovery mixture was plated on LB agar plates containing ampicillin at concentration 100 μg/ml. Plates were incubated overnight at 37° C. Colonies were screened by colony PCR for the presence of the insert. The sequences of the genes were confirmed. Strain designation and genetic constructs are in Table 10.

TABLE 10

| Designation | Construct | Gene |
|---|---|---|
| pMx0308 | pGAN-ALAsynth | ALA synthase |
| pMx0309 | pGAN-ALAD | ALAD |
| pMx0310 | pGAN-UPGIIIsyn | Uroporphyrinogene |
| | | synthase |
| pMx0311 | pGAN-UPGIIIdecarb | Uroporphyrinogene |
| | | decarboxylase |
| pMx0312 | pGAN-CPGoxi | CPG oxidase |
| pMx0313 | pGAN-PPGoxi | Protoporphyrin oxidase |
| pMx0314 | pGAZ-ALAsyn | ALA synthase |
| pMx0315 | pGAZ-ALAD | ALAD |
| pMx0316 | pGAZ-UPGIIIsyn | Uroporphyrinogene |
| | | synthase |
| pMx0317 | pGAZ-UPGIIIdecarboxilase | UPGIII decarboxilase |
| pMx0318 | pGAZ-PPGoxidase | PPG oxidase |
| pMx0319 | pGAZ-CPG oxidase | CPG oxidase |
| pMx0320 | pGAN-PBD | PBD |
| pMx0321 | pGAZ-PGC | PBD |
| pMx0322 | pGAZ-Fc | Ferrochelatase |
| pMx0323 | pGAN-Fc | Ferrochelatase |

Example 12—Assembling Heme Biosynthesis Genes on Plasmids for Integration into P. Pastoris mutS Genome

The whole cassette “promoter-gene-terminator” was PCR amplified with primers containing sites for restriction endonucleases to assemble plasmids for integration into the Pichia genome.

Assembling Paox1_UPS_FDHterm-Paox1_UPD_FDHterm-Paox1_CPO_FDH term. on pGAN plasmid (pMx327)

pGAN-CPGoxidase (pMx312) was used as a vector to clone the UPS and UPD cassettes. UPG III synthase cassette was PCR amplified from pMx310 with primers to AOX1 promoter/FDH1 terminator containing Nhel and SphI recognition sites for restriction endonucleases correspondingly (Table 11):

TABLE 11

| Primer | Recognition | | SEQ ID |
|---|---|---|---|
| Designation | Sites | Sequence | NO: |
| MxO0399 | NheI-pAOX1-F | CAA TCG CTA GCA TCC | 31 |
| | | AAC ATC CAA AGA CGA | |
| | | AAG G | |
| MxO0401 | SphI-FDH1-R | GGA TAG CAT GCA CCT | 32 |
| | | TAT CAA GAT AGC TAG | |
| | | AAA TAG AAA TGG | |

UPG III decarboxylase cassette was PCR amplified from pMx311 with primers to AOX1 promoter/FDH1 terminator containing SphI and Agel recognition sites for restriction endonucleases correspondingly (Table 12):

TABLE 12

| | | | SEQ |
|---|---|---|---|
| Primer | Recognition | | ID |
| Designation | Sites | Sequence | NO: |
| MxO0402 | SphI- | CAA TAG CAT GCA ACA TCC | 33 |
| | pAOX1I-F | AAA GAC GAA AGG TTG AAT | |
| | | G | |
| MxO0404 | AgeI-FDH1-R | CAT GGT ACC GGT ACC TTA | 34 |
| | | TCA AGA TAG CTA GAA ATA | |
| | | GAA ATGG | |

PHUSION® High-Fidelity DNA Polymerase (New England BioLabs, Cat #M0530L) was used to amplify DNA from plasmids.

Obtained PCR products were purified using DNA Clean&Concentrator-5 (Zymo Research, Cat #D4004) and DNA was eluted in 25 μl of H₂O.

pGAN-CPGoxidase (pMx312) designated as a vector was digested in 1x CUTSMART® Buffer with NheI-HF (New England BioLabs, Cat #R3131S) and AgeI-HF (New England BioLabs, Cat #R3552S) over night at 37° C.

UPG III synthase cassette PCR product was digested in 1x CUTSMART® Buffer with NheI-HF (New England BioLabs, Cat #R3131S) and SphI-HF (New England BioLabs, Cat #R3182S) over night at 37° C.

UPG III decarboxylase cassette PCR product was digested in 1× CUTSMART® Buffer with SphI-HF (New England BioLabs, Cat #R3182S) and AgeI-HF (New England BioLabs, Cat #R3552S) over night at 37° C.

Digested vector and PCR products were gel purified from 0.8% agarose using ZYMOCLEAN™ Gel DNA Recovery Kit (Zymo Research, Cat #D4002). DNA was eluted in 20 μl H₂O.

Three way ligation between UPG III synthase cassette digested with NheI-SphI, UPG III decarboxylase cassette digested with SphI-AgeI and a vector digested with NheI-AgeI was set up in 10 μl at 16° C. overnight using T4 DNA Ligase (New England BioLabs, Cat #M0202S).

1.5 μl of ligation mixture was transformed into 20 μl of ElectroMax DH10B T1Phage-Resistant Competent Cells (Invitrogen, Cat #12033-015) by electroporation using MICROPULSERTM (BioRad) set at 1.7 kV; cells were incubated at 37° C. in 1 ml SOC for 1 h with shaking at 200 rpm. 10 μl of recovery mixture was plated on LB agar plates containing ampicillin at a concentration of 100 μg/ml. Plates were incubated overnight at 37° C. Colonies were screened by colony PCR for the presence of the insert. The sequences of the junctions between vector and inserts were confirmed.

Assembling Paox1_ALAsynthase_FDH1term.-Paox1_PPGoxidase_FDHIterm-Paox1_Fc_FDHIterm.-Paox1_PBD_ FDHIterm cassette (pMx330)

### A. PCR Amplification of Gene-Cassettes:

ALAsynthase cassette was PCR amplified from pMx310 with primers to AOX1 promoter/FDH1 terminator containing NheI and XhoI recognition sites for restriction endonucleases correspondingly (Table 13):

TABLE 13

| Primer | Recognition | | SEQ ID |
|---|---|---|---|
| Designation | Sites | Sequence | NO: |
| MxO0399 | NheI-pAOX1-F | CAA TCG CTA GCA TCC | 35 |
| | | AAC ATC CAA AGA CGA | |
| | | AAG G | |
| MxO0400 | XhoI-FDH1-R | GAT ATT GCT CGA GAC | 36 |
| | | CTT ATC AAG ATA GCT | |
| | | AGA AAT AGA AAT G | |

PPGoxidase cassette was PCR amplified from pMx313 with primers to AOX1 promoter/FDH1 terminator containing XhoI and Af1II recognition sites for restriction endonucleases correspondingly (Table 14):

TABLE 14

| Primer | Recognition | | SEQ ID |
|---|---|---|---|
| Designation | Sites | Sequence | NO: |
| MxO0403 | XhoI-pAOX1-F | CAA TCT CGA GAA CAT | 37 |
| | | CCA AAG ACG AAA GGT | |
| | | TG | |
| MxO0437 | AflII-FDH1-R | CAA CCA TTT CTA TTT | 38 |
| | | CTA GCT ATC TTG ATA | |
| | | AGG TCT TAA GTC CA | |

Ferrochelatase cassette was PCR amplified from pMx323 with primers to AOX1 promoter/FDH1 terminator containing Af1II and AgeI recognition sites for restriction endonucleases correspondingly (Table 15):

TABLE 15

| | | | SEQ |
|---|---|---|---|
| Primer | Recognition | | ID |
| Designation | Sites | Sequence | NO: |
| MxO0404 | AgeI-FDH1-R | CAT GGT ACC GGT ACC | 39 |
| | | TTA TCA AGA TAG CTA | |
| | | GAA ATA GAA ATG G | |
| MxO0436 | AflII-pAOX1-F | TTA CTT AAG TCC AAC | 40 |
| | | ATC CAA AGA CGA AAG | |
| | | GTT G | |

G418 marker was PCR amplified from pJAG plasmid purchased from Biogrammatics using the following primers (Table 16):

TABLE 16

| | | | SEQ |
|---|---|---|---|
| Primer | Recognition | | ID |
| Designation | Sites | Sequence | NO: |
| MxO0438 | Mlu-G418-F | TCA CAG ACG CGT TGA | 41 |
| | | ATT GTC C | |
| MxO0439 | BbvCI-G418-R | TTG CTC CTC AGC TTA | 42 |
| | | GAA GAA CTC GTC CAA | |
| | | CAT CAA GTG | |

PHUSION® High-Fidelity DNA Polymerase (New England BioLabs, Cat #M0530L) was used to amplify DNA from plasmids. The PCR products were obtained and purified using DNA Clean&Concentrator-5 (Zymo Research, Cat #D4004) and DNA was eluted in 25 μl of H₂O.

b. Preparation of Vectors

pGAZ-PBD (pMx321) designated as a vector was digested in 1x CUTSMART® Buffer with NheI-HF (New England BioLabs, Cat #R3131S) and Xhol (New England BioLabs, Cat #R0146S) overnight at 37° C.

pGAZ-ALAsyn.-PBD (pMx328) was digested in 1× NEBuffer3.1 with MluI (New England BioLabs, Cat #R0198S) and BbvCI (New England BioLabs, Cat #R0601S) overnight at 37° C.

pGAG-ALAsyn-PBD (pMx332) was digested in 1× CUTSMART® Buffer with Xhol (New England BioLabs, Cat #R0146S) and AgeI-HF (New England BioLabs, Cat #R3552S) overnight at 37° C.

c. Making Intermediate Constructs and Assembling a Final Cassette

Digested vector and PCR products were gel purified from 0.8% agarose using ZYMOCLEAN™ Gel DNA Recovery Kit (Zymo Research, Cat #D4002). DNA was eluted in 20 μl of H₂O.

pGAZ-PBD (pMx321) vector, digested with NheI-XhoI restriction endonucleases, was ligated with ALAsynthase cassette PCR product digested with the same enzymes in 10 μl reaction at 16° C. overnight using T4 DNA Ligase (New England BioLabs, Cat #M0202S) to yield a pGAZ-ALAsyn.-PBD plasmid (pMx328).

pGAG-ALAsyn-PBD (pMx332) digested with Xhol and AgeI-HF restriction endonucleases was ligated with PPGoxidase cassette and Ferrochelatase cassette PCR products digested with XhoI, AflII and AflII, AgeI-HF restriction endonucleases correspondingly in a three way ligation reaction using T4 DNA Ligase (New England BioLabs, Cat #M0202S) to yield a pGAG-ALAsynthase_PPGoxidase_Fc_PBD (pMx330).

1.5 μl of ligation mixture was transformed into 20 μl of ElectroMax DH10B TI Phage-Resistant Competent Cells (Invitrogen, Cat #12033-015) by electroporation using MICROPULSER™ (BioRad) set at 1.7 kV; cells were incubated at 37° C. in 1 ml SOC for 1 h with shaking at 200 rpm. 10 μl of recovery mixture was plated on LB agar plates containing ampicillin at a concentration of 100 μg/ml. Plates were incubated overnight at 37° C. Colonies were screened by colony PCR for the presence of the insert. The sequences of junctions between the vector and the inserts were confirmed. Strain designations and genetic constructs are described in Table 17.

TABLE 17

| Designation | Construct | Gene |
|---|---|---|
| pMx0327 | pGAN-UPGsyn_ | UPGsyn_UPGdecarb_ |
| | UPGdecarb_CPGoxy | CPGoxy |
| pMx0328 | pGAZ-ALAsyn-PGC | ALAsyn-PBD |
| pMx0330 | pGAG-ALAsyn_PBD_PPG_Fc | ALAsyn_PBD_PPG_Fc |
| pMx0332 | pGAG-ALAsyn_PBD | G418 marker |

Example 13—Integration of Linearized Plasmids with Gene Cassettes into P. Pastoris Bg11 Genome

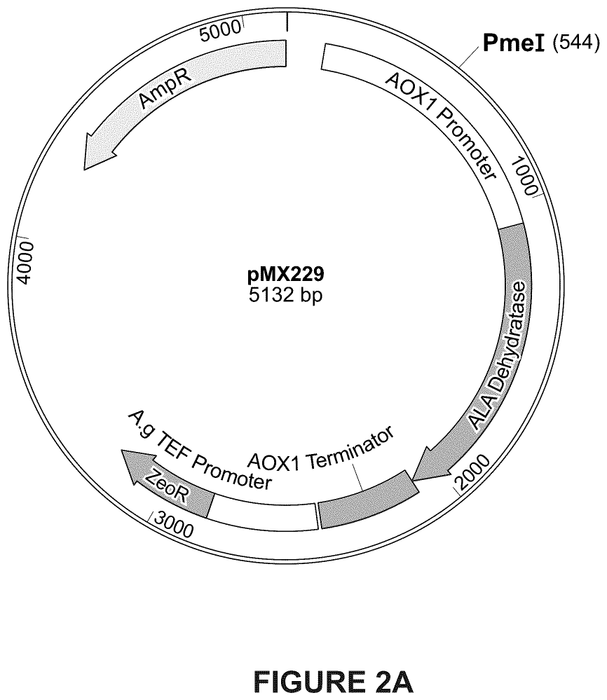

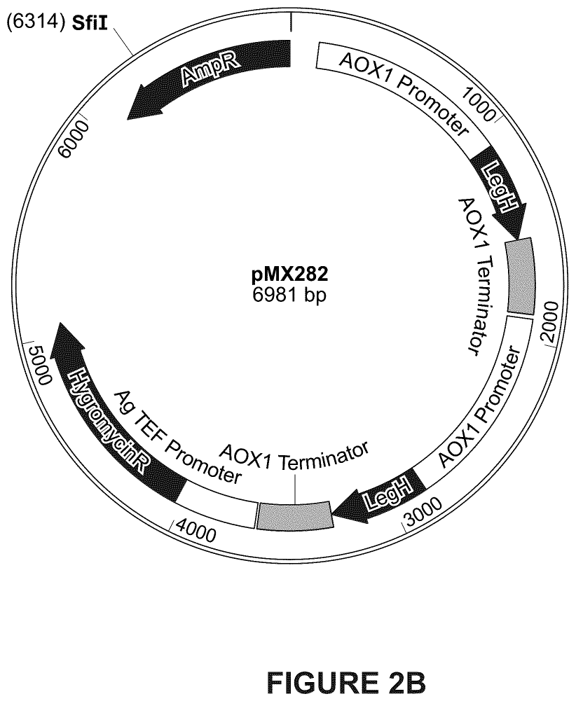

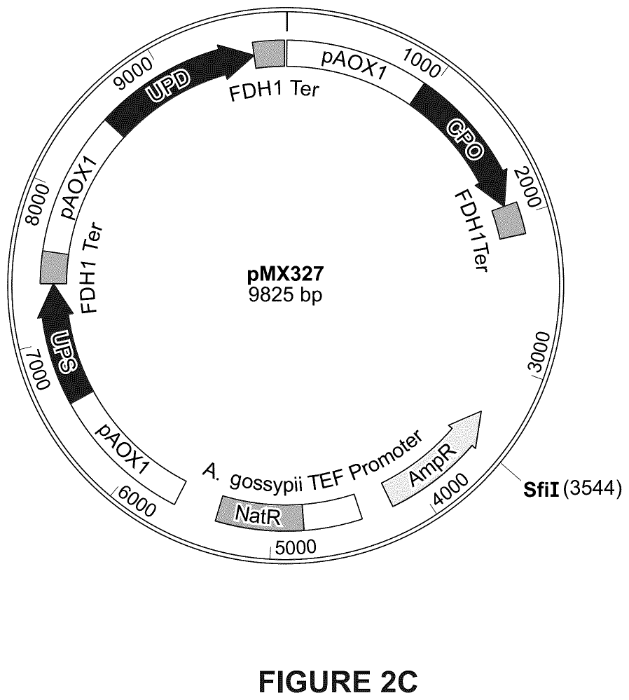

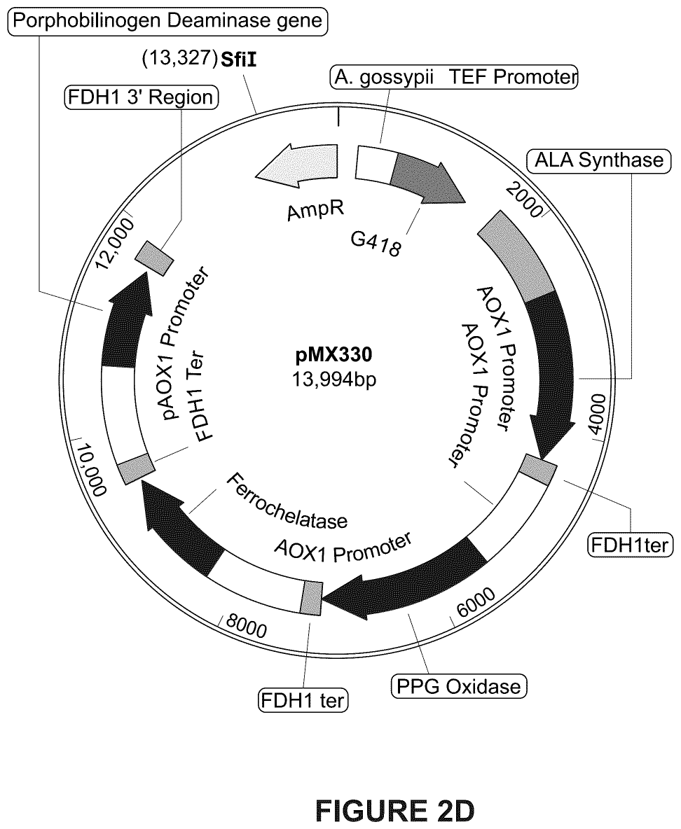

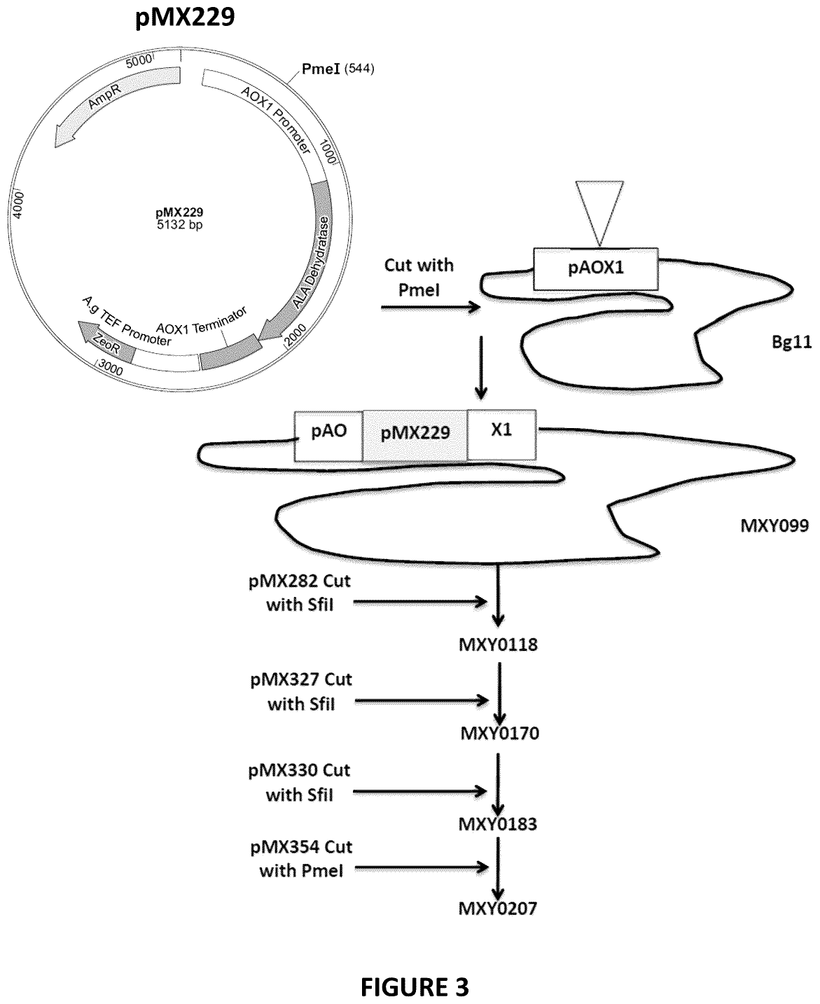

The plasmids that were used to generate the production strain, MXY0183, are shown in FIG. 2. The steps taken to make the modifications that led to production strain, MXY0183, are depicted in FIG. 3.

The first enzyme to be introduced into the P. pastoris Bg11 genome was ALAD. A plasmid containing pAOX1-driven ALAD (pMX229, FIG. 2i and FIG. 3) was linearized using PmeI restriction enzyme (New England BioLab). Linearized plasmid was purified from 0.8% agarose gel as described and transformed into P. pastoris using homologous recombination at the native AOX1 locus, generating strain MXY099 (FIG. 3).

A plasmid containing two copies of the soybean LegH gene (sequence optimized for P. pastoris; SEQ ID NO:3) under the control of the pAOX1 promoter designated pMX282 (FIG. 2ii and FIG. 3) was linearized using SfiI restriction enzyme. Linearized plasmid was purified from a 0.8% agarose gel as described and transformed into the P. pastoris strain containing ALAD, generating the strain MXY0118 (FIG. 3). qPCR was used and determined that strain MXY0118 contained several copies of the LegH gene, likely due to concatamerization of the plasmid, pMX282, at the time of recombination.

Plasmid pMX327 (FIG. 2iii and FIG. 3) containing genes encoding Uroporphyrinogen III synthase (UPS), Uroporphyrinogen III decarboxylase (UPD) and Coproporphyrinogen III oxidase (CPO) (the enzymes catalyzing steps 4, 5 and 6, respectively) under control of the AOX1 promoter was linearized with the Sfil restriction endonuclease and introduced into MXY0118, yielding strain MXY0170 (FIG. 3).

Genes encoding ALA synthase (ALAS), Protoporphyrin III oxidase (PPO), Ferrochelatase (FC) and Porphobilinogen deaminase (PBD) (the enzymes catalyzing steps 1, 7, 8 and 3, respectively) from the P. pastoris genome were assembled on plasmid pMX330 (FIG. 2iv and FIG. 3). pMX330 was linearized with the Sfil restriction endonuclease and transformed into MXY0170, leading to the generation of strain MXY0183 (FIG. 3). The genotype of MXY0183 was confirmed using PCR and qPCR.

### Production Strain MXY0207

Example 14—Construction of pGAB Expression Vector

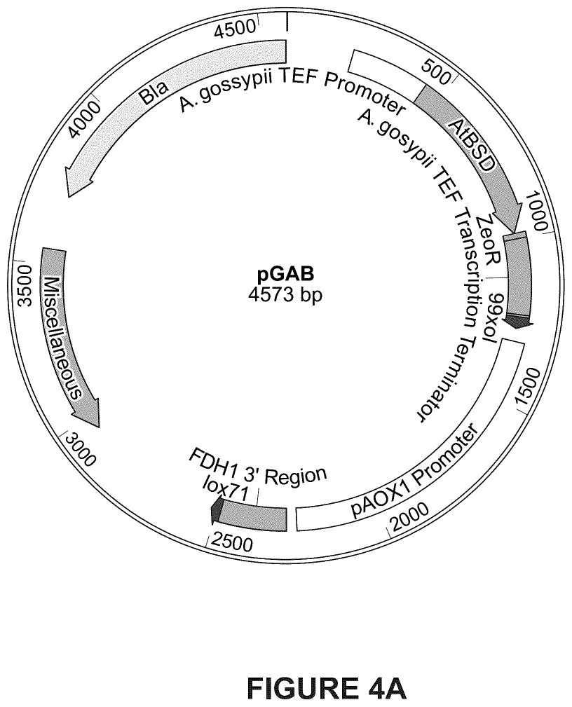

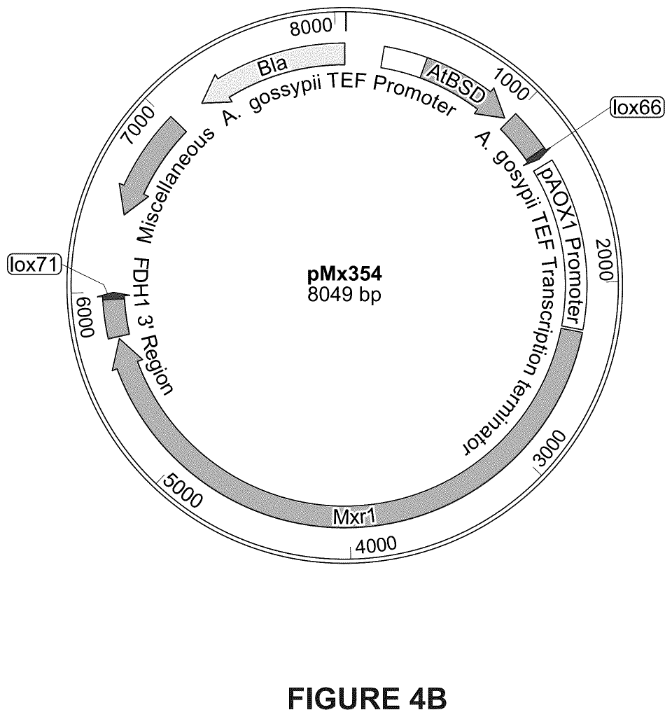

The pGAB expression vector (FIG. 4A) was constructed by replacing the open reading frame of the ZEOCIN™ resistance gene in the pGAZ vector (BioGrammatics, Inc., Carlsbad, CA) with the open reading frame from the Blasticidin S deaminase (BSD) gene from Aspergillus terreus, which allows for selection of transformants carrying the plasmid with the antibiotic Blasticidin S.

The BSD open reading frame was amplified from a commercially synthesized DNA molecule using oligonucleotide primers Mx00476 and Mx00477 using a high fidelity polymerase chain reaction as described herein (Table 18).

TABLE 18

| | | | SEQ |
|---|---|---|---|
| Primer | | | ID |
| Designation | Description | Sequence | NO: |
| MxO0477 | BSD_Reverse | TTA GTC TTG CTC CTC | 43 |
| | | AGC TTA GCC | |
| MxO0476 | BSD_Forward | TCA CAG ACG CGT TGA | 44 |
| | | ATT GTC C | |

The BSD PCR product was purified by gel electrophoresis on a 1% agarose gel in 1× TBE buffer (89 mM Tris, 89 mM boric acid, 2 mM EDTA, pH 8.3) and visualized using SYBR™ Safe DNA gel stain (Life Technologies, Carlsbad, CA). The desired DNA fragment was excised from the agarose gel and the DNA was recovered using the ZYMOCLEAN™ Gel DNA Recovery Kit (Zymo Research, Irvine, CA).

The purified BSD PCR product and pGAZ vector were digested with 10 units each of the MluI and BbvCI restriction endonucleases (New England Biolabs, Ipswich, MA) for 1 hour at 37° C. in 1× NEBuffer 3.1 (100 mM NaCl, 50 mM Tris-HCl, 10 mM MgCl₂, 100 μg/ml BSA, pH 7.9@25° C.). Digested DNA products were recovered by gel electrophoresis as described above.

The purified, MluI and BbvCI digested BSD product and pGAZ vector were incubated with 400 units of T4 DNA ligase (New England Biolabs) in 1× T4 DNA ligase reaction buffer (50 mM Tris-HCl, 10 mM MgCl₂, 1 mM ATP, 10 mM DTT, pH 7.5 @25° C.) in a 20 μl reaction, at 16° C. for 2 hours in a 20 μl reaction. Electrocompetent E. coli DH10B cells were transformed with 2 μl of the ligation reaction and antibiotic resistant transformants were selected on LSB agar plates supplemented with 100 μg/μl ampicillin.

### Example 15—Construction of Mxr1 Expression Vector

The Mxr1 expression vector, pMx354, was constructed by introducing the Mxr1 open reading frame into the pGAB vector (FIG. 4B). The Mxr1 open reading frame was inserted into pGAB with the translation start immediately downstream of the methanol-inducible alcohol oxidase 1 (AOX1) promoter from Pichia pastoris and the translation stop signal immediately followed by the transcription terminator sequence from the P. pastoris FDH1 gene.

The open reading frame encoding the Mxr1 protein was amplified from genomic DNA isolated from Pichia pastoris strain Bg11 MutS obtained from BioGrammatics, Inc. (Carlsbad, CA). The Mxr1 open reading frame was amplified from P. pastoris genomic DNA with primers Mx00495 (TTT TGC GGC CGC ATG AGC AAT CTA CCC CCA ACT TTT G (SEQ ID NO:45)) and Mx00496 (AAA AGC GGC CGC CTA GAC ACC ACC ATC TAG TCG GTT (SEQ ID NO:46)), which appended flanking Notl restriction endonuclease recognition sites. Amplification was accomplished using the polymerase chain reaction as described herein.

The amplified Mxr1 PCR product and the pGAB vector were digested with 10 units of NotI restriction endonuclease (New England Biolabs) for 1 hour at 37° C. in 1× NEBuffer 3.1 (100 mM NaCl, 50 mM Tris-HCl, 10 mM MgCl_{2, 100} μg/ml BSA, pH 7.9 @25° C.). Following digestion, the NotI-digested pMx352 vector was treated with 5 units Antarctic phosphatase (New England Biolabs) for 15 minutes at 37° C. in 1× Antarctic phosphatase buffer (50 mM Bis-Tris-Propane-HCl, 1 mM MgCl₂, 0.1 mM ZnCl₂, pH 6 @ 25° C.).

The NotI-digested amplified Mxr1 fragment and pMx352 vector were separated by electrophoresis on a 1% agarose gel in 1× TBE buffer (89 mM Tris, 89 mM boric acid, 2 mM EDTA, pH 8.3) and visualized using SYBR™ Safe DNA gel stain (Life Technologies, Carlsbad, CA). The desired DNA fragments were excised from the agarose gel and the DNA was recovered using the ZYMOCLEANTM Gel DNA Recovery Kit (Zymo Research, Irvine, CA).

The NotI-digested fragment containing Mxr1 open reading frame was introduced into pGAB at a NotI site immediately downstream of the AOX1 promoter by ligation. A mixture containing 137 ng of NotI-digested DNA encoding the Mxr1 open reading frame and 60 ng of NotI-digested, phosphatase-treated pMx352 was incubate with 400 units of T4 DNA ligase (New England Biolabs) in 1× T4 DNA ligase reaction buffer (50 mM Tris-HCl, 10 mM MgCl₂, 1 mM ATP, 10 mM DTT, pH 7.5 @ 25° C.) in a 20 μl reaction, at 16° C., for 2 hours in a 20 μl reaction. Electrocompetent E. coli DH10B cells were transformed with 2 μl of the ligation reaction and antibiotic resistant transformants were selected on LSB agar plates supplemented with 100 μ/μl ampicillin. Plates were incubated overnight at 37° C. Colonies were screened for the presence of the insert by PCR using primers Mx00495 and Mx00496. The sequence of the final vector was confirmed by DNA sequencing.

During cloning, 6 additional amino acids were introduced at the N-terminus of Mxr1. The Mxr1 open reading frame is shown under the section “Nucleic acid sequences”, with residual amino acids from the cloning shown with underlining. Pichia production strains containing the Mxr1 sequence having the additional 6-amino acids at the N-terminus and Pichia strains containing the wild type Mxr1 (i.e., without the additional 6 amino acids at the N-terminus) were indistinguishable in fermentation tanks.

### Example 16—Construction of Native Mxr1 Expression Vector

A plasmid containing the Mxr1 transcription regulator gene under the control of the pAOX1 promoter, designated pMX354, was used as a template for PCR amplification. The 3′ end of the AOX1 promoter, the LegH open reading frame, and the AOX1 terminator were amplified from pMX354 using primers Mx00617 and Mx00647 shown below. The AOX1 terminator, linker and the 5′ end of the AOX1 promoter were amplified from pMX382 using primers Mx00618 and Mx00646 (Table 19).

TABLE 19

| | | SEQ |
|---|---|---|
| Primer | | ID |
| Designation | Sequence | NO: |
| MxO0646 | ACTAGATGGTGGTGTCTAGTCAAGAGGATGTC | 47 |
| | AGAATGCCATTTG | |
| MxO0647 | TCTGACATCCTCTTGACTAGACACCACCATCT | 48 |
| | AGTCGGTTTTCTAG | |

PCR products were obtained and purified using DNA Clean&Concentrator-5 and DNA was eluted in 12 μl of H₂O. The purified PCR products were then combined and used as a template for a subsequent round of PCR amplification using primers Mx00617 and Mx00618. The resulting PCR product was composed of the 3′ end of the AOX1 promoter, followed by the Mxr1 open reading frame, the AOX1 terminator, a short linker sequence, and the 5′ end of the AOX1 promoter. The PCR product was obtained and purified as described herein. The purified PCR product was cloned into the pCR™-Blunt II-TOPO® vector using the Zero Blunt® TOPOR PCR Cloning Kit (Invitrogen, Cat #K2800-20) to create the pMX402 vector.

### Example 17—Construction of P. Pastoris Strains MXY0206 and MXY0207

The pMx354 Mxr1 expression vector (FIG. 4B) was introduced into the MXY0183strain by DNA transformation (FIG. 3).

The pMx354 vector (1.5 μg) was linearized at a unique PmeI site in the AOX1 promoter sequences by digestion with 20 units of the PmeI restriction endonuclease (New England Biolabs) for 1 hour at 37° C. in 1× NEBuffer 4 (50 mM Potassium Acetate, 20 mM Tris-acetate, 10 mM Magnesium Acetate, 1 mM DTT, pH 7.9@25° C.).

The PmeI-digested pMX354 vector was purified by gel electrophoresis recovered using the ZYMOCLEAN™ Gel DNA Recovery Kit as described above. The linearized pMX354 vector was introduced into strain MXY0183 by transformation and selection on blasticidin-containing medium. Two independent clones were obtained from the transformation, and they were designated MXY0206 and MXY0207. The presence of an additional copy of Mxr1 under the control of the AOX1 promoter in these strains was confirmed by PCR.

### Production Strain MXY0291

### Example 18—Construction of Strains MXY0213 and MXY0260

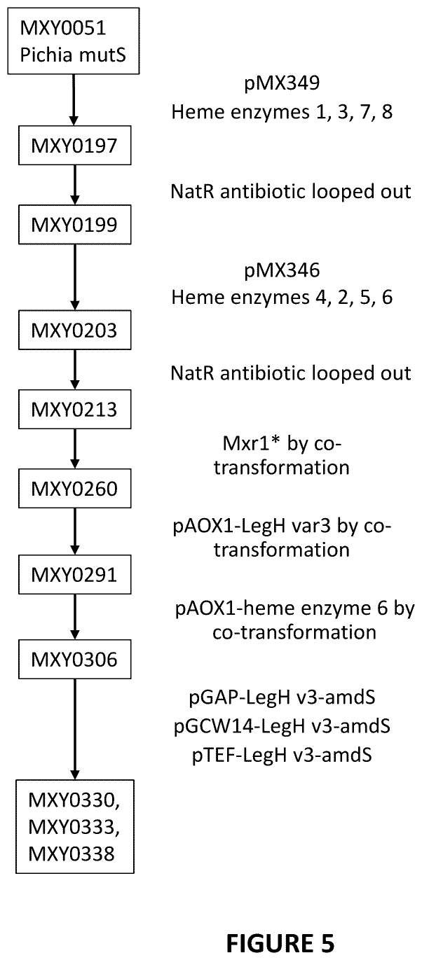

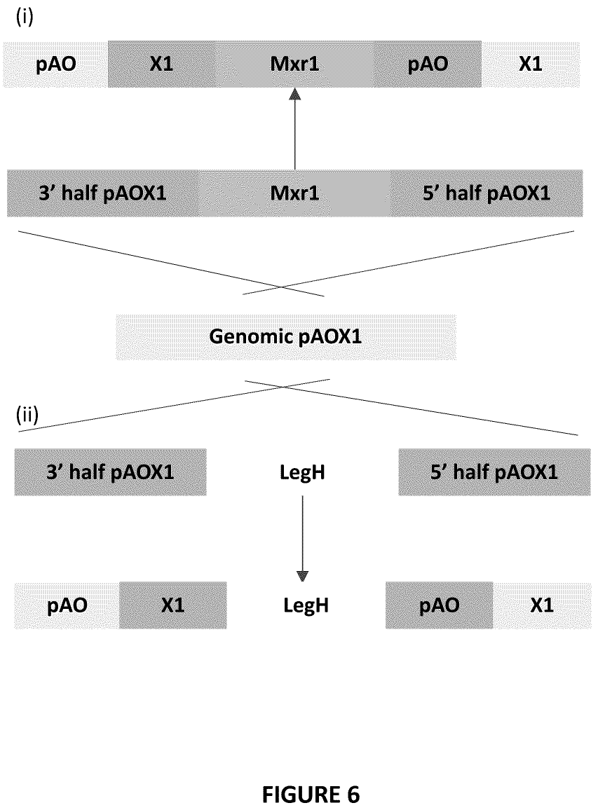

FIG. 5 shows the steps taken to construct antibiotic marker free strain MXY0213 that contains 7 enzymes of the heme biosynthetic pathway. A linear piece of DNA containing variant Mxr1 (6 extra amino acids at N terminus) under pAOX1, with homology to the pAOX1 promoter on each end, was introduced using co-transformation (FIG. 5 and FIG. 6i). This linear Mxr1 expression cassette was simultaneously introduced into Pichia strain MXY213 with the pIL75 plasmid by transformation. The pIL75 vector carries a panARS autonomous replication sequence (Liachko & Dunham, 2014, FEMS Yeast Res., 14:364-7), which allows for maintenance of the plasmid vector without integration into the genome of the transformed cells, and a kanMX marker for selection of transformants with the antibiotic G418. Transformed cells were selected on media supplemented with G418 for the presence of kanMX marker on the pIL75 plasmid. Pichia transformants were screened by colony PCR for transformants that took up both the pIL75 plasmid and had correctly integrated the Mxr1 expression cassette.

### Example 19—Co-Transformation to Introduce the LegH Expression Cassette Into Pichia

A plasmid containing a different Pichia pastoris-codon optimized variant of soybean LegH gene (variant 3; SEQ ID NO:5) under the control of the pAOX1 promoter designated pMX399 was used as a source of template for PCR amplification of the gene. The backbone from TOPO cloning plasmid pMX401 was PCR amplified. The insert and vector were assembled using GIBSON ASSEMBLY® (NEB GIBSON ASSEMBLY® kit) to generate plasmid pMX422. This plasmid was used as a template for a subsequent round of PCR amplification using primers Mx00617 and Mx00618 shown below (Table 20).

TABLE 20

| | | SEQ |
|---|---|---|
| Primer | | ID |
| Designation | Sequence | NO: |
| MxO0617 | AAACGCTGTCTTGGAACCTAATATGAC | 49 |
| MxO0618 | AAACTGTCAGTTTTGGGCCATTTG | 50 |

The resulting PCR product was composed of, in the 5′ to 3′ direction, the 3′ end of the AOX1 promoter, followed by the LegH var 3 open reading frame, the AOX1 terminator, a short linker sequence, and the 5′ end of the AOX1 promoter (FIG. 6ii). The PCR product was obtained and purified by agarose gel electrophoresis as described herein.

Transformants with LegH expression cassette integrated into the genome were screened by PCR and characterized for LegH gene copy number using qPCR.

Example 20—Curing Transformants of Plasmid Vectors Bearing Selection Markers In clones where the soybean LegH expression cassette was shown to be correctly integrated by colony PCR and in high copy number by qPCR, the pIL75 plasmid required for selection on G418 was eliminated by relaxing selection for the antibiotic. Transformants were streaked out for single colonies on media lacking G418 antibiotic. Because the panARS plasmid is not stably maintained in the absence of selection, the pIL75 was rapidly lost from the transformed cells under this condition. The resulting Pichia strain, MXY0291, contains sequences for LegH expression in copy number similar to MXY0207, but lacks heterologous sequences for selection.

### Production Strains MXY0330, MXY0333, and MXY0338

### Example 21—Construction of Strain MXY0306

Genotype PCR of strain MXY0291 revealed that a portion of the CPGoxidase coding sequence had been deleted during construction of this strain. The full-length CPGoxidase coding region was restored by replacement of the truncated copy. Briefly, a linear DNA fragment containing the pAOX1 promoter and full-length CPGoxidase coding region was generated by PCR amplification from plasmid pMX312 using primers Mx00866 and Mx00867 shown below (Table 21).

TABLE 21

| Primer | | SEQ ID |
|---|---|---|
| Designation | Sequence | NO: |
| MxO0866 | ACGCTGTCTTGGAACCTAATATGAC | 51 |
| MxO0867 | TACCCATTCAATAGGATTTTGTAGTACCTGC | 52 |

The linear pAOX1-CPGoxidase DNA fragment was introduced into strain MXY0291 by co-transformation with the pIL75 plasmid. Transformants were selected on media containing G418 and then screened for the presence of the full-length CPGoxidase coding region by PCR. An isolate containing the full-length CPGoxidase was identified and subsequently cured of the plasmid vector required for selection on G418 as described above. This strain was designated MXY0306 (see FIG. 5).

### Example 22—Linear Constructs for Hybrid Promoter Strains

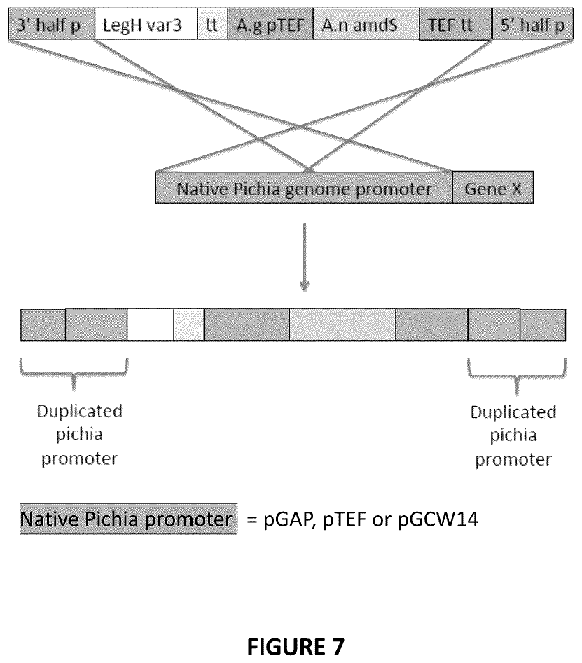

LegH variant 3 was expressed under the direction of each of the three native Pichia pastoris constitutive promoters indicated herein. The linear constructs are shown in FIG. 7, and contained the 3′ half of the promoter, followed by LegH var3, followed by the FDH1transcription terminator. This was immediately followed by the antibiotic resistance cassette containing the pTEF promoter from Ashbya gossypii, the acetamidase gene (amdS) from Aspergillus nidulans and the TEF terminator from Ashbya gossypii. Finally, the construct contained the 5′ half of the promoter. This linear cassette was amplified using the oligonucleotide primers listed in Table 22 below to generate constructs that contain several hundred base pairs on the 5′ and 3′ ends that are homologous to the respective promoter in the native Pichia genome.

TABLE 22
Primers used to amplify the linear constructs

| Primer | | SEQ |
|---|---|---|
| designation | Sequence | ID NO: |
| MXO0718 | GAGCTTCTTCTACGGCCCCC | 53 |
| MXO0723 | TCCAGCAGAGTAAAATTTCCTAGGGAC | 54 |
| MXO0724 | CTCTTTTAGGTTTTAAGTTGTGGGAACAGTA | 55 |
| | ACA | |
| MXO0729 | GTGGGTGCTTCTTTGCGTGG | 56 |
| MXO0730 | AGAATTGCCATCAAGAGACTCAGGACT | 57 |
| MXO0735 | GATAGAGAGAAATCGCAAACTTTGAGAGGAA | 58 |
| | G | |

Competent MXY0306 cells were transformed with each of the linear cassettes and transformants containing the amdS selection cassette were selected based on their ability to grow on agar plates containing acetamide as the sole nitrogen source. These strains were purified, isolated and the presence of LegH under control of the constitutive promoter was verified by PCR (FIG. 5).

### Example 23—Nucleic Acid Sequences

Mxr1 nucleic acid sequence (the underlined nucleotides encode 6
amino acid at N-term introduced during cloning)
(SEQ ID NO: 1)
ATGCGAGACCGCGGCCGCATGAGCAATCTACCCCCAACTTTTGGTTCCACTAGACAATCTCCAGA
AGACCAATCACCTCCCGTGCCCAAGGAGCTGTCATTCAATGGGACCACACCCTCAGGAAAGCTAC
GCTTATTTGTCTGTCAGACATGTACTCGAGCATTTGCTCGTCAGGAACACTTGAAACGACACGAA
AGGTCTCACACCAAGGAGAAACCTTTCAGCTGCGGCATTTGTTCTCGTAAATTCAGCCGTCGAGA
TCTGTTATTGAGACATGCCCAAAAACTGCACAGCAACTGCTCTGATGCGGCCATAACAAGACTAA
GGCGCAAGGCAACTCGTCGGTCTTCTAATGCCGCGGGTTCCATATCTGGTTCTACTCCGGTGACA
ACGCCAAATACTATGGGTACGCCCGAAGATGGCGAGAAACGAAAAGTTCAGAAACTGGCCGGCCG
CCGGGACTCAAATGAACAGAAACTGCAACTGCAACAACAACATCTACAGCAACAACCACAGTTGC
AATACCAACAATCTCTTAAGCAGCATGAAAATCAAGTCCAGCAGCCTGATCAAGATCCATTGATA
TCCCCGAGAATGCAATTATTCAATGATTCCAACCATCACGTAAACAATTTGTTTGATCTTGGACT
AAGAAGAGCTTCCTTCTCCGCCGTTAGTGGAAATAATTATGCCCATTATGTGAATAATTTTCAAC
AAGATGCCTCTTCTACCAATCCAAATCAAGATTCAAATAATGCCGAATTTGAGAATATTGAATTT
TCTACCCCACAAATGATGCCCGTTGAAGATGCTGAAACTTGGATGAACAACATGGGTCCAATTCC
GAACTTCTCTCTCGATGTGAACAGGAACATTGGTGATAGCTTTACAGATATACAACACAAGAATT
CAGAGCCTATTATATCCGAACCGCCCAAGGACACCGCTCCAAACGACAAGAAGTTGAATGGCTAC
TCTTTTTACGAAGCCCCCATCAAGCCATTAGAATCCCTATTTTCTGTCAGGAATACAAAGAGAAA
CAAGTATAAAACAAATGACGACTCTCCAGACACCGTGGATAATAACTCCGCACCGGCTGCTAATA
CCATTCAAGAACTTGAGTCTTCTTTGAATGCATCCAAGAATTTTTGCTTGCCAACTGGTTATTCC
TTCTATGGTAATTTGGACCAACAGACTTTCTCTAACACGTTATCATGCACTTCTTCTAATGCCAC
AATTTCGCCCATTCTACTCGATAACTCCATTAATAATAACTCCACTAGTGACGTGAGACCAGAAT
TTAGAACACAAAGTGTCACCTCTGAAATGAGTCAAGCCCCTCCCCCTCCTCAAAAAAACAACTCG
AAATATTCCACCGAAGTTCTTTTTACCAGCAACATGCGGTCGTTTATTCACTACGCTCTTTCCAA
GTATCCTTTTATTGGTGTGCCCACTCCAACTCTTCCGGAGAACGAAAGACTAAATGAATATGCTG
ATTCATTCACCAACCGTTTCTTAAATCATTATCCTTTCATACATGTCACGATTCTCAAAGAATAC
TCCCTTTTCAAGGCAATTTTAGATGAGAATGAGTCGACTAAGAACTGGGAAAATAATCAGTTTTA
CTTAGAGAACCAACGAATATCAATTGTTTGTCTTCCTCTTTTGGTGGCTACGATAGGTGCAGTAC
TATCAAACAACAAAAAGGATGCTTCGAATTTATACGAAGCTTCAAGGCGTTGTATTCATGTTTAC
TTAGATTCCAGGAAAAAGATACCCACTTCCTTGTCCGCAAATAACAATGACTCTCCACTTTGGCT
AATTCAATCCCTGACGTTATCTGTTATGTATGGGTTATTTGCGGACAATGACATTAGTTTGAATG
TCGTGATCAGACAAGTTAACGCACTTAATTCTCTGGTCAAGACTTCGGGCCTGAATAGGACCTCA
ATTATAGATCTTTTCAACATCAACAAACCTTTGGATAATGAACTCTGGAATCAATTCGTGAAAAT
AGAGTCCACCGTAAGGACAATCCACACGATTTTTCAAATCAGTTCCAACTTAAGCGCCTTGTACA
ATATTATTCCATCGTTGAAAATTGATGACCTAATGATTACTCTACCAGTTCCCACAACACTTTGG
CAAGCTGATTCTTTTGTGAAATTCAAAAGTCTAAGTTACGGAAATCAGATCCCTTTTCAATATAC
AAGAGTACTACAGAATTTGATTGATTACAATCAGCCATTGAGCGATGGAAAATTTTTGTATGAAA
ACCATGTAAGTGAGTTTGGACTCATATGCCTACAGAATGGTCTACACCAATACAGCTATTTCCAA
AAATTGACTGCTGTCAATAACAGAGAAGATGCGCTATTCACAAAGGTTGTTAATTCACTTCACAG
TTGGGATAGGATGATTTCGAATTCTGATTTGTTTCCAAAGAAGATATATCAGCAGAGTTGCTTGA
TTTTGGACTCAAAGTTGCTTAATAATTTCCTGATTGTCAAGAGCTCATTGAAAGTTTCGACCGGA
GACGTTAGTTCTTTGAATAAGTTAAAAGAAAACGTGTGGCTTAAAAACTGGAATCAAGTGTGTGC
TATCTATTATAACAGCTTCATGAACATTCCTGCTCCCAGTATTCAAAAGAAGTACAATGACATAG
AGTTTGTGGATGACATGATTAATTTGAGTCTAATCATCATCAAGATTATGAAACTCATTTTCTAT
AACAATGTCAAAGACAATTATGAGGATGAAAATGACTTCAAATTGCAAGAGTTAAATTTAACATT
TGACAATTTTGATGAGAAAATATCCTTGAATTTGACAATATTATTCGATATATTTTTGATGATCT
ACAAGATAATTACCAATTACGAAAAGTTTATGAAGATCAAACACAAGTTTAATTACTACAATTCT
AATTCGAATATAAGCTTCTTGCATCATTTCGAACTCTCCTCGGTTATCAATAACACCCAAATGAA
CCAGAATGATTATATGAAAACAGATATTGATGAAAAGCTTGATCAGCTTTTCCACATCTATCAAA
CATTTTTCCGGCTGTATCTGGATTTAGAAAAGTTTATGAAGTTCAAATTCAACTATCATGACTTT
GAGACAGAGTTTTCAAGTCTCTCAATATCCAATATACTGAACACTCATGCTGCTTCTAACAATGA
CACAAATGCTGCTGATGCTATGAATGCCAAGGATGAAAAAATATCTCCCACAACTTTGAATAGCG
TATTACTTGCTGATGAAGGAAATGAAAATTCCGGTCGTAATAACGATTCAGACCGCCTGTTCATG
CTGAACGAGCTAATTAATTTTGAAGTAGGTTTGAAATTTCTCAAGATAGGTGAGTCATTTTTTGA
TTTCTTGTATGAGAATAACTACAAGTTCATCCACTTCAAAAACTTAAATGACGGAATGTTCCACA
TCAGGATATACCTAGAAAACCGACTAGATGGTGGTGTCTAG
Mxr1 protein sequence (the underlined 6 amino acid at N-term
introduced during cloning)
(SEQ ID NO: 2)
MRDRGRMSNLPPTFGSTRQSPEDQSPPVPKELSFNGTTPSGKLRLFVCQTCTRAFARQEHLKRHE
RSHTKEKPFSCGTCSRKFSRRDLLLRHAQKLHSNCSDAATTRLRRKATRRSSNAAGSTSGSTPVT
TPNTMGTPEDGEKRKVQKLAGRRDSNEQKLQLQQQHLQQQPQLQYQQSLKQHENQVQQPDQDPLT
SPRMQLFNDSNHHVNNLFDLGLRRASFSAVSGNNYAHYVNNFQQDASSTNPNQDSNNAEFENTEF
STPQMMPVEDAETWMNNMGPTPNFSLDVNRNTGDSFTDTQHKNSEPTTSEPPKDTAPNDKKLNGY
SFYEAPTKPLESLFSVRNTKRNKYKTNDDSPDTVDNNSAPAANTTQELESSLNASKNFCLPTGYS
FYGNLDQQTFSNTLSCTSSNATTSPTLLDNSTNNNSTSDVRPEFRTQSVTSEMSQAPPPPQKNNS
KYSTEVLFTSNMRSFTHYALSKYPFTGVPTPTLPENERLNEYADSFTNRFLNHYPFTHVTTLKEY
SLFKATLDENESTKNWENNQFYLENQRTSTVCLPLLVATTGAVLSNNKKDASNLYEASRRCTHVY
LDSRKKTPTSLSANNNDSPLWLTQSLTLSVMYGLFADNDTSLNVVTRQVNALNSLVKTSGLNRTS
TTDLFNTNKPLDNELWNQFVKTESTVRTTHTTFQTSSNLSALYNTTPSLKTDDLMTTLPVPTTLW
QADSFVKFKSLSYGNQTPFQYTRVLQNLTDYNQPLSDGKFLYENHVSEFGLTCLQNGLHQYSYFQ
KLTAVNNREDALFTKVVNSLHSWDRMTSNSDLFPKKTYQQSCLTLDSKLLNNFLTVKSSLKVSTG
DVSSLNKLKENVWLKNWNQVCATYYNSFMNTPAPSTQKKYNDTEFVDDMTNLSLTTTKTMKLTFY
NNVKDNYEDENDFKLQELNLTFDNFDEKTSLNLTTLFDTFLMTYKTTTNYEKFMKTKHKFNYYNS
NSNTSFLHHFELSSVTNNTQMNQNDYMKTDTDEKLDQLFHTYQTFFRLYLDLEKFMKFKFNYHDF
ETEFSSLSTSNTLNTHAASNNDTNAADAMNAKDEKTSPTTLNSVLLADEGNENSGRNNDSDRLFM
LNELTNFEVGLKFLKTGESFFDFLYENNYKFTHFKNLNDGMFHTRTYLENRLDGGV*
Pichia pastoris-Codon-optimized LegH nucleic acid sequence
(SEQ ID NO: 3)
ATGGGTGCTTTCACCGAGAAGCAGGAAGCACTTGTTTCCTCTTCGTTCGAAGCTTTTAAGGCTAA
CATCCCTCAATACTCTGTTGTGTTTTACACGTCCATTCTAGAAAAAGCTCCTGCTGCCAAGGACC
TCTTCTCTTTTCTGTCCAACGGTGTAGATCCATCCAATCCCAAATTAACAGGTCACGCTGAGAAA
TTGTTCGGTTTAGTCAGAGATAGCGCTGGACAATTGAAAGCAAATGGTACTGTGGTTGCTGATGC
TGCCTTGGGCAGCATCCATGCACAGAAGGCAATTACAGACCCACAATTTGTTGTTGTGAAGGAAG
CTCTGCTTAAAACTATAAAGGAAGCCGTCGGAGACAAATGGAGTGACGAGTTGTCATCAGCTTGG
GAGGTAGCTTATGATGAGTTGGCCGCAGCAATCAAAAAGGCATCTAA
Pichia pastoris-Codon-optimized LegH amino acid sequence
(SEQ ID NO: 4)
MGAFTEKQEALVSSSFEAFKANIPQYSVVFYTSILEKAPAAKDLFSFLSNGVDPSNPKLTGHAEK
LFGLVRDSAGQLKANGTVVADAALGSIHAQKAITDPQFVVVKEALLKTIKEAVGDKWSDELSSAW
EVAYDELAAAIKKAF
Pichia pastoris-Codon-optimized LegH variant 3 nucleic acid
sequence
(SEQ ID NO: 5)
ATGGGTGCATTTACAGAAAAACAAGAGGCTTTAGTATCCTCATCTTTTGAAGCTTTCAAAGCCAA
TATTCCTCAATACTCCGTTGTTTTCTATACGTCCATTTTGGAAAAGGCTCCAGCAGCTAAGGACC
TTTTCTCTTTCTTGTCGAACGGCGTGGATCCCTCAAATCCTAAGCTGACTGGTCACGCCGAGAAG
CTTTTTGGTTTGGTCAGAGACAGCGCCGGACAGCTGAAAGCTAACGGTACAGTTGTGGCAGATGC
TGCCTTGGGATCTATACATGCACAAAAGGCTATCACCGACCCACAGTTTGTGGTTGTAAAAGAGG
CTCTACTCAAAACTATCAAGGAAGCAGTTGGTGACAAATGGAGCGATGAATTGTCCAGTGCATGG
GAGGTCGCTTACGATGAGTTAGCTGCTGCAATCAAAAAGGCTTTCTAA
Pichia pastoris-Codon-optimized LegH variant 3 amino acid
sequence
(SEQ ID NO: 6)
MGAFTEKQEALVSSSFEAFKANIPQYSVVFYTSILEKAPAAKDLFSFLSNGVDPSNPKLTGHAEK
LFGLVRDSAGQLKANGTVVADAALGSIHAQKAITDPQFVVVKEALLKTIKEAVGDKWSDELSSAW
EVAYDELAAAIKKAF
Pichia pastoris pAOX1 promoter
(SEQ ID NO: 7)
GATCTAACATCCAAAGACGAAAGGTTGAATGAAACCTTTTTGCCATCCGACATCCACAGGTCCAT
TCTCACACATAAGTGCCAAACGCAACAGGAGGGGATACACTAGCAGCAGACCGTTGCAAACGCAG
GACCTCCACTCCTCTTCTCCTCAACACCCACTTTTGCCATCGAAAAACCAGCCCAGTTATTGGGC
TTGATTGGAGCTCGCTCATTCCAATTCCTTCTATTAGGCTACTAACACCATGACTTTATTAGCCT
GTCTATCCTGGCCCCCCTGGCGAGGTTCATGTTTGTTTATTTCCGAATGCAACAAGCTCCGCATT
ACACCCGAACATCACTCCAGATGAGGGCTTTCTGAGTGTGGGGTCAAATAGTTTCATGTTCCCCA
AATGGCCCAAAACTGACAGTTTAAACGCTGTCTTGGAACCTAATATGACAAAAGCGTGATCTCAT
CCAAGATGAACTAAGTTTGGTTCGTTGAAATGCTAACGGCCAGTTGGTCAAAAAGAAACTTCCAA
AAGTCGGCATACCGTTTGTCTTGTTTGGTATTGATTGACGAATGCTCAAAAATAATCTCATTAAT
GCTTAGCGCAGTCTCTCTATCGCTTCTGAACCCCGGTGCACCTGTGCCGAAACGCAAATGGGGAA
ACACCCGCTTTTTGGATGATTATGCATTGTCTCCACATTGTATGCTTCCAAGATTCTGGTGGGAA
TACTGCTGATAGCCTAACGTTCATGATCAAAATTTAACTGTTCTAACCCCTACTTGACAGCAATA
TATAAACAGAAGGAAGCTGCCCTGTCTTAAACCTTTTTTTTTATCATCATTATTAGCTTACTTTC
ATAATTGCGACTGGTTCCAATTGACAAGCTTTTGATTTTAACGACTTTTAACGACAACTTGAGAA
GATCAAAAAACAACTAATTATTCGAAACG
Pichia pastoris pGAP promoter
(SEQ ID NO: 8)
CGACTATTATCGATCAATGAAATCCATCAAGATTGAAATCTTAAAATTGCCCCTTTCACTTGACA
GGATCCTTTTTTGTAGAAATGTCTTGGTGTCCTCGTCCAATCAGGTAGCCATCTCTGAAATATCT
GGCTCCGTTGCAACTCCGAACGACCTGCTGGCAACGTAAAATTCTCCGGGGTAAAACTTAAATGT
GGAGTAATGGAACCAGAAACGTCTCTTCCCTTCTCTCTCCTTCCACCGCCCGTTACCGTCCCTAG
GAAATTTTACTCTGCTGGAGAGCTTCTTCTACGGCCCCCTTGCAGCAATGCTCTTCCCAGCATTA
CGTTGCGGGTAAAACGGAGGTCGTGTACCCGACCTAGCAGCCCAGGGATGGAAAAGTCCCGGCCG
TCGCTGGCAATAATAGCGGGCGGACGCATGTCATGAGATTATTGGAAACCACCAGAATCGAATAT
AAAAGGCGAACACCTTTCCCAATTTTGGTTTCTCCTGACCCAAAGACTTTAAATTTAATTTATTT
GTCCCTATTTCAATCAATTGAACAACTATCAAAACACG
Pichia pastoris pGCW14 promoter
(SEQ ID NO: 9)
CAGGTGAACCCACCTAACTATTTTTAACTGGGATCCAGTGAGCTCGCTGGGTGAAAGCCAACCAT
CTTTTGTTTCGGGGAACCGTGCTCGCCCCGTAAAGTTAATTTTTTTTTCCCGCGCAGCTTTAATC
TTTCGGCAGAGAAGGCGTTTTCATCGTAGCGTGGGAACAGAATAATCAGTTCATGTGCTATACAG
GCACATGGCAGCAGTCACTATTTTGCTTTTTAACCTTAAAGTCGTTCATCAATCATTAACTGACC
AATCAGATTTTTTGCATTTGCCACTTATCTAAAAATACTTTTGTATCTCGCAGATACGTTCAGTG
GTTTCCAGGACAACACCCAAAAAAAGGTATCAATGCCACTAGGCAGTCGGTTTTATTTTTGGTCA
CCCACGCAAAGAAGCACCCACCTCTTTTAGGTTTTAAGTTGTGGGAACAGTAACACCGCCTAGAG
CTTCAGGAAAAACCAGTACCTGTGACCGCAATTCACCATGATGCAGAATGTTAATTTAAACGAGT
GCCAAATCAAGATTTCAACAGACAAATCAATCGATCCATAGTTACCCATTCCAGCCTTTTCGTCG
TCGAGCCTGCTTCATTCCTGCCTCAGGTGCATAACTTTGCATGAAAAGTCCAGATTAGGGCAGAT
TTTGAGTTTAAAATAGGAAATATAAACAAATATACCGCGAAAAAGGTTTGTTTATAGCTTTTCGC
CTGGTGCCGTACGGTATAAATACATACTCTCCTCCCCCCCCTGGTTCTCTTTTTCTTTTGTTACT
TACATTTTACCGTTCCGTCACTCGCTTCACTCAACAACAAAA
Pichia pastoris pTEF1 promoter
(SEQ ID NO: 10)
ATAACTGTCGCCTCTTTTATCTGCCGCACTGCATGAGGTGTCCCCTTAGTGGGAAAGAGTACTGA
GCCAACCCTGGAGGACAGCAAGGGAAAAATACCTACAACTTGCTTCATAATGGTCGTAAAAACAA
TCCTTGTCGGATATAAGTGTTGTAGACTGTCCCTTATCCTCTGCGATGTTCTTCCTCTCAAAGTT
TGCGATTTCTCTCTATCAGAATTGCCATCAAGAGACTCAGGACTAATTTCGCAGTCCCACACGCA
CTCGTACATGATTGGCTGAAATTTCCCTAAAGAATTTcTTTTTCACGAAAATTTTTTTTTTACAC
AAGATTTTCAGCAGATATAAAATGGAGAGCAGGACCTCCGCTGTGACTCTTCTTTTTTTTCTTTT
ATTCTCACTACATACATTTTAGTTATTCGCCAAC
Heme biosynthesis enzyme 1-ALA Synthase
(SEQ ID NO: 11)
ATGGAGTTTGTCGCCCGTCAGTCCATGAATGCCTGTCCCTTTGTCAGGTCAACTTCTACCCACCA
TTTGAAGAAGTTGGCAGCAAACAGTTCTCTAGCTGCTACTGCTAGTCATTGTCCCGTGGTTGGCC
CTGCTCTCCAACAGCAGAGATACTACTCTCAACCTTCCAAGCCAGCCCAAGCCCAAACCTCCGAC
ATTGCTACTGGGATCAAGAAGGATGTTTCTCCGATCCGTATGGACTCTAATGAAACCGCCTTTGA
TTACAATGGAATGTATGAGTCTGATCTTGCGAATAAACGTAAAGATAACTCGTATCGTTATTTCA
ATAACATCAACCGTCTAGCCAAGGAGTTTCCCAAGGCACATCGCCAGACCGAAGATGACAAGGTG
ACCGTCTGGTGCTCTAACGACTACTTAGGAATGGGTAGGCATCCTGAGATTATCAAAACCATGAA
GGCTACCATGGACAAGTACGGTTCCGGAGCAGGAGGAACTAGGAACATTGCAGGTCATAACCACG
CCGCTATCAATTTGGAAAGCGAGTTGGCTTGCTTGAACAAGAAGGAAGCGGCTCTGGTGTTTTCA
TCATGTTTCATAGCTAACGATGCAATCATCTCGTTGTTGGGACAAAAAATCAAAAATTTGGTCAT
TTTCTCTGACCAGTCGAATCATGCTTCCATGATATTGGGTGTGCGTAACTCCAAAGCGAAGAAGC
ACATCTTCAAGCACAACAATTTGAAGGATCTGGAGTCGCAGTTAGCTCAGTACCCCAAGTCGACT
CCTAAACTGATCGCCTTCGAGTCAGTTTACTCTATGTGTGGATCTGTGGCTCCCATTGAGAAGAT
TTGCGATTTGGCTAAAAGGTACGGTGCCCTCACCTTCTTGGATGAAGTTCATGCTGTTGGAATGT
ATGGTCCTCATGGACAGGGTGTAGCTGAGCATTTGGACTTTGATCTGCATTTACAGTCTGGAATC
GCCAGTCCTAGCGTGGTGGACAAACGCACCATATTGGATCGTGTCGACATGATTACTGGTACTTG
CGGAAAGTCATTTGGTACTGTTGGAGGTTACGTTGCTGGTAGTGCCAACCTAATTGATTGGTTAA
GATCCTATGCGCCAGGTTTCATTTTCACTACCACACTTCCTCCTGCTATCATGGCTGGTACAGCC
ACTTCTGTTCGTATTGTTAGGGCCGACATTGAGGCCCGTATCAAGCAACAGCTTAATACTCGCTA
CGTCAAAGACTCATTTGAAAACCTTGGTATTCCAGTCATTCCAAACCCAAGTCACATTGTTCCTG
TTCTAGTTGGAAATGCTGCAGATGCCAAGAAGGCATCCGATATGTTAATGAACAAACACCGTATT
TATGTTCAAGCTATTAACTACCCTACTGTGCCTGTCGGTGAAGAACGACTAAGGATTACTCCTAC
TCCAGGTCATGGAAAGGAGATTTGTGACCAGCTGATCAGCGCTGTCGACGATGTTTTTACTGAGC
TTAATTTACCAAGAATCAACAAATGGCAGTCCCAAGGTGGTCATTGCGGTGTTGGTGATGCTAAT
TACGTACCAGAACCCAATCTGTGGACTCAGGACCAGCTCAGCTTGACAAACCAAGACTTGCACTC
CAATGTGCACAACCCAGTGATTGAGCAGATCGAAACCTCATCAGGAGTCAGATTGTAG
Heme biosynthesis enzyme 2-ALA dehydratase
(SEQ ID NO: 12)
ATGGTGCATAAGGCTGAATACTTGGACGACCACCCAACTCAGATTTCCAGCATTCTTTCAGGAGG
TTACAACCACCCATTACTTCGTGAATGGCAACATGAACGTCAACTCAACAAAAACATGTTCATCT
TTCCCCTGTTTGTCACAGATCGACCAGACGAAGAAGAACTTATTCCTAGTCTACCTAATATCAAG
AGGTTTGGCGTTAACAAGTTGATTCCTTATGTAGGAGGTTTGGTTTCCAAAGGATTGAGGGCGGT
GATCCTATTTGGTGTTCCTCTGAAGCCCGGTGTGAAAGATGAAGAAGGAACGGCCGCTGATGATC
CAGAGGGACCTGTTATCCAAGCCATCAAACACTTGAGAAAGAACTTTCCTGACCTGTATATCATC
ACCGATGTCTGTCTATGTGAGTACACCAGCCATGGACATTGTGGAATACTATATGAGGATGGCAC
TATCAACAGAGAGCTCTCAGTCCGTCGTATTGCTGCTGTAGCTGTCAAATATGCTCAAGCTGGAG
CCAACTCTGTGGCTCCTTCTGATATGACTGACGGCAGAATAAGAGATATTAAAGAAGGCTTACTA
AGTGCAGGACTGGCACATAAAACGTTTGTTATGTCCTACGCTGCAAAATTCTCTGGTAATTTGTA
TGGCCCTTTCAGAGATGCTGCAGGTTCCTGTCCATCTCAAGGGGACAGAAAATGTTACCAGCTTC
CTTCTGGAGGAAAAGGGTTGGCCCATCGTGCTCTGATTCGTGATATGAATGAAGGCACTGATGGA
ATTATTGTCAAACCATCTACATTCTATTTGGACATTGTCGCTGATGCTTATCAGCTTTGTAAAGA
CTATCCTATCTGCTGTTACCAGGTTTCTGGAGAGTACGCCATGCTACATGCAGCGGCAGAGAAGA
ATATTGTTGATCTGAAATCAATCGCTTTTGAAGCTCATCAAGGATTCTTGCGGGCTGGAGCTCGT
TTAATCATTAGTTACTTTACCCCTGAATTCCTGGAGTGGTTATCTGAATGA
Heme biosynthesis enzyme 3-Porphobilinogen deaminase
(SEQ ID NO: 13)
ATGAACCAAATCGAACAGAGCGGACCCATTGATTGCAGTTCCTTGAAATTGGGGTCCCGAAAGTC
CGCTCTGGCTATAATCCAGGCAGAAATCGTCCGCCAATTGATATTGAAAGAATACCCTGAATTGG
AGACGAAGTTGGTCAGTGTGTCCACCCTGGGGGACCAAGTCCAGAATAAAGCACTTTTCACGTTT
GGAGGAAAATCTTTGTGGACCAAAGAACTTGAGATGTTGTTGTTGGAGAGTGTGGGAGGATTTGA
CCAAATAGACATGATTGTACACTCGTTGAAAGACATGCCAACTCATTTACCAGACGAATTTGAGC
TGGGTTGCATTATTGAAAGAGAAGACCCTAGAGACGCTTTGGTCGTGCAAGATGGTTTATCTTAC
AAGTCATTGGCCGACCTTCCAGAGGGAGCTGTGGTCGGTACGTCTTCGGTTAGAAGATCGGCTCA
ACTACTGAAGAATTTCCCTCATCTGAAATTCAAATCTGTTAGAGGAAACCTTCAGACCAGACTAA
GAAAATTAGATGATCCAGATTCCGAGTACTGCTGTCTCCTCCTTGCAGCAGCCGGTTTAATCAGG
ACAGGCTTACAACACAGAATTTCAATGTATTTGAACGACGATGTGATGTACCACTCCGTCGGACA
AGGAGCATTAGGAGTAGAGATCAGAAAAGGTGACCAATTCATGAAAAATATCTGTGAAAAGATTG
GGCATAGAACCACCACCCTTCGTTGTCTTGCAGAGAGAGCACTGCTGAGATATCTAGAGGGAGGC
TGCTCGGTGCCAATTGGGGTCTCCACTATTTATAGCGAGGATACGAAGGAACTTACCATGAACTC
CCTAGTCGTCAGTTGTAACGGTCGTGACTCGGTAACAGAATCAATGACTGAAGTCGTGACTACTG
AAGAGCAAGCTGAAGATTTCGGTGAAAGGCTGGCCCAGAAGCTCATAGATCAAGGTGCGAAACGC
ATTCTTGACGAGATCAACTTCAACAAGATCAAAGAGATTAAGGAAGAGGGTTTACATTAA
Heme biosynthesis enzyme 4-Uroporphyrinogen III synthase
(SEQ ID NO: 14)
ATGCCAAAAGCCATTCTTCTGAAGAATAAAACTACACCGAAGGATCCTTATCTGGAGAACTTCGT
AAGTAGTGGCTACTCGACCGATTTCGTACCACTTTTAGATCATATTCACATGGAGAAATCTGAGA
TCATCGCATTTCTCAAGACTGACTACTTTTTGCATAAAACTTTGGCGTTTATTATTACGTCCCAA
AGAGCTGTAGAAATGCTGAATGAGTGTATGCAAATACTGAGACGTACTGATCCTGAAATTACACA
AATCATCTATAGTAAACCTGTCTATACAGTTGGCCCTGCCACCTACAGAATACTTGCGGATGCTG
GCTTCGTGGATCTACGAGGCGGAGATAAGGCAGGAAACGGATCCATTCTAGCCCAGATAATTTTG
AATGATGACATTTACACTGGAATTGAAGATTCTGACAAGCATATAACGTTTTTCACGGGAGAAAC
AAGGAGAGACATAATTCCCAAATGTTTACTCTCTAACAACTTTCAACTTTACGAAAAGATTGTCT
ACAAGACTCTTCCTAGGGATGATATCGTGACTAGATTCAAGTCTGCCGTTGACAGCATCGACCAA
TCGCAAAGAAGTTCCAGTTGGGTGGTCTTCTTTTCGCCTCAAGGAACAGAGGACATTGTAACGTA
TCTTCAACACACCAAAGACCAATTTAATATTGCATCTATCGGGCCAACCACAGAAAAATACCTTC
TAAGCAAAAACCTGAAACCAAAAGTTGTGGCACCTAAGCCAGAGCCTATCTCTTTACTATTGTCT
ATACAAAAAGTGCACTAA
Heme biosynthesis enzyme 5-Uroporphyrinogen III decarboxylase
(SEQ ID NO: 15)
ATGAGTAGATTTCCAGAACTGAAGAATGACCTTATTTTAAGGGCAGCTCGTGGTGAAAAAGTTGA
ACGTCCCCCAATATGGATTATGAGACAGGCCGGAAGATATCTTCCGGAGTACCATGAGGTCAAAG
GAGGTAGGGACTTCTTTGAAACTTGCAGGGATGCTGAGATTGCTTCTGAAATTACTATCCAGCCG
ATTACGCATTTTGACGGTCTGATCGATGCAGCTATTATCTTCAGTGATATCTTGGTGATTCCTCA
AGCTATGGGCATGGAAGTTAAGATGGTGGACAAAGTTGGCCCACAGTTCCCCAATCCGCTAAGAA
AACCGTCTGACTTGGATCATTTGAAAAAAGACGTTGACGTTTTGAAGGAACTCGATTGGGCCTTC
AAAGCTATCTCATTGACCAGAAAAAAACTCAATGGACGAGTGCCTTTGCTTGGATTTTGTGGTGC
TCCTTGGACTCTACTGGTTTATATGACTGAAGGAGGCGGTACCAAGATGTTTCGATTTGCAAAAG
AGTGGATCTACAAGTTTACCAAGGAATCTCATCAATTACTCCAACAGATCACTGACGTTGCAGTT
GAATTCTTAGCTCAGCAAGTTGTTGCAGGTGCCCAAATGTTACAAGTTTTTGAATCTTGGGGCGG
TGAATTGGGGCCTGATGAATTCGATGAGTTTTCTTTGCCTTATTTGAGACAGATTTCCTCTAAAC
TTCCCCTGAGGTTGAAGGAACTTGGAATCACAGAGAATGTTCCCATAACTGTCTTTGCTAAAGGC
TCTTGGTACGCCTTGGAGCAATTGTGCGACAGTGGTTATGATGTTGTCTCGTTGGATTGGTTATT
CCGTCCAAGTGATGCTGTCCAGATTGCTAACGGAAGAATCGCATTGCAAGGTAATCTTGACCCTG
GAACCATGTACGGCTCCAAAGAAACCATTTCCAAGAAAGTGGACAAAATGATCAAGGGTTTTGGT
GGAGGAAAGCAAAACTACATAATTAATTTTGGACACGGCACTCATCCATTCATGGATCCAGAACA
GATCAGATGGTTCTTACAAGAATGTCATCGCATTGGATCTCAATAG
Heme biosynthesis enzyme 6-Coproporphyrinogen III oxidase
(SEQ ID NO: 16)
ATGGCCATCGACTCTGATATCAATCTAAGCTCTCCCAATGATTCCATCCGTCAAAGGATGTTCGA
GCTTATCCAGCGGAAGCAACTCGAAATTGTCGCTGCATTGGAGGCAATTGAAGGAAACGATACCA
ATTTCGTTCTGATTCTTGGGAAAGAGGAGCCGAAGGTGGAGGAGGAAGATCTATGCTTATTCAA
GATGGAAGAGTGTTTGAAAAGGCTGGTGTAAATATTTCCAAGGTTCATGGCGTATTGCCTCCTCA
AGCTGTGAGCCAGATGAGAAATGACCACTCCAAGCTAGATCTGCCTGCGGGAACCTCTCTGAAGT
TCTTTGCCTGTGGGCTTTCGTTGGTCATTCATCCCCATAATCCCCATGCTCCAACTACCCATCTG
AATTATCGCTACTTCGAAACTTGGGATGAAACTGGAAAGCCTCACACCTGGTGGTTTGGGGGCGG
TGCTGATTTAACGCCTTCGTACCTGTATCCCGAGGATGCCAAGCAATTCCATCAAGCCCATAAGG
ATGCCCTGGACAAACACGATGTTAGCTTGTACCCGAGATTCAAAAAGTGGTGTGATGAATACTTT
CTGATCAAACATCGAAATGAAACTAGAGGTATTGGGGGTATTTTCTTTGATGATTTTGACGAGTT
TGATGCTGAGAGGTCCCTGAAGTTGGTTGAAGATTGTTTCAATGCTTTCTTGGAATCTTATCCCG
CTATCACTCGAAAAAGGATGGACACCCCTTCAACTGATGCTGAGAAGAACTGGCAACAAATTAGA
AGAGGAAGATATGTCGAATTCAACTTAGTATTGGATAGAGGTACTCAATTTGGTTTGAGAACGCC
TGGATCTCGTGTTGAAAGTATTTTGATGTCGTTGCCAAGAACAGCTGGTTGGGTCTATGATCATC
ATCCAGAGCCTGGCTCCAGAGAAGAGGAGTTATTGCAGGTACTACAAAATCCTATTGAATGGGTA
TGA
Heme biosynthesis enzyme 7-Protoporphyrinogen oxidase
(SEQ ID NO: 17)
ATGCTGAAAAGTCTTGCACCAAATTCCTCAATTGCCGTTTTAGGTTCAGGGATATCTGGATTGAC
TTTCAGCTTTTTTTTGAATCGGTTGCGTCCCGATGTTAAGATCCATATCTTTGAAAAATCCAAGC
AGGTTGGAGGATGGATCAGATCAGAAGAGCATGAAACTTTTCATTTTGAAAAGGGACCCAGAACT
TTGAGAGGCACAAATACGGGTACCTTGATGTTGTTGGATCTTCTTACCAAGATAGGAGCAAATGA
CAAGGTCCTGGGACTGCACAAAGATTCTCTTGCTAATAAAAAGTATCTGTTGTCCCCGTTCTCAG
ATGTTCACGGAAACAACGCAAAGCTTCTTCAAGTGCCACAGGATTTCAGCTCTTTTGTAAAGTTC
ATGTTTGACCCGTTGTCTAAGGATCTCATTCTCGGTCTTTTGAAAGAACCATGGCAACCAAAATT
AAAGTATTCAGATGAGTCGGTTGACCATTTTTTCAACAGAAGATTTGCTACCAAACTATCAGAGA
ATATCGTCAGCGCAATTGTGCATGGAATCTATGCGGGCGACGTGAAGAAGTTAAGTGTGAAAGCC
ATCTTCCCTAGGCTCCCTGAGATGGAACAGGAAAGTGGCTCTATTATAAGGTATATGATCGCCCA
ATACAGGACAAAAAAGAACGTCAAACAAAAAGTTGACCCTTTTTTGGCAGATTATGAAAAATTGA
TCGGTACATCTTTGAGTTTCAAAAATATTTCTTTGTTTCTGAAAAACTTTCCCATGCTGAGTTTT
CAGGGTGGACTACAGAAACTTCCCATCTCATTGAAGAACCATTTATCACAGATTGAAAACATCAA
GTTTCATTTTGACAGCAAAATCAAAAACATTGCTTTGGAGAGCGGTAAGGTGGCATTGACTGACC
ATGATCAGGTTTATCTTGTTGACCATGTGAGATCTACCATTAATACCAACGAATTGGCCAAAATC
ATTTCACCCGTTGTTCCAAGTTCTACTAAGAAAAAATCCGTTTTCAAATCCAAAGCGAATGGCCC
AGGGCTGGTCAAATGTTTGAGCTGGCTACACTATACAAATATACTAATGTGCAACATTTATATAC
CTAAGCACGTCTCAAAATCTATCACCGGATTTGGATACTTGGTTCCTCGATCAATGTCTTCTCAG
GCATCCAAACTTCTCGGTGTCATATTTGACTCAGACATCGAGACTGCAATGACTCCTAATTTTAC
AGAGGCCAACATTACGGCGATAAACAGTAACTCTGCATCTCCCAAGCAACTCCAAAAGTTTTCTG
ACCAATTCGTCAATAATGATCTCCCTAAATACACCAAGTTGACGCTAATGCTTGGAGGTCATTAT
CTCAAGTCGGAGGCAGACATGCCCGGTTCCGCAGAGAGTAAACATGCTGTCAAGGCGATTCTGTC
AAATCACCTGAATATTGATCTAGATGAGTTTGCATCTTTGCCAGACTTCAAGATGGAAATCACCA
AGATCCCCAACTGCATTCCCCAATATGAAGTTGGGTATCTTGATCTCAAGAGAAAGGTTCAGAAT
GCAGCCTCCAAAGAGTTCAACGACCAAATAAGTTTTGGAGGCATGGCATTTGGTGATGGTGTGGG
GATCCCTGACTGTGTCCAGAATGCATTCAAAGATTCGGCTACCCTCAGTGGCATTTAA
Heme biosynthesis enzyme 8-Ferrochelatase
(SEQ ID NO: 18)
ATGCTTAACCGTCGTTTCCAATCTACCGTGTCCTCGAGTCTGAACAAGGGCACTGGAATAGTGTT
CATGAATATGGGTGGTCCCTCCACTGTCAAGGAAACCTATGACTTTTTATTTCGTCTTTTCTCGG
ACGGAGATTTAATCCCGTTTGGCAGATTTCAGAACATCCTGGCCCGCTTCATTGCAAGTAGAAGA
ACACCCAAAATTGAATCCTACTACAAAGCTATCGGAGGTGGGTCTCCTATCCGAAAGTGGTCTGA
ATACCAGAGTTCTAAACTATGTGAAAAATTAGACATTATCAGTCCACAATCGGCTCCTCATAAGC
CTTATGTTGCCTTCAGATACGCTAATCCTCTCACTGAAGATACTTTACAAAAGATGAAAAATGAT
GGAATTACTAAGGCCATTGCCTTTTCTCAATATCCGCAATTTAGTTATTCAACCACCGGATCATC
GATTAACGAACTTTACAGGCAATCGAAAATTTTGGACCCTGATCAATCTATTAAATGGACAGTTA
TAGATCGCTGGCCTGACCACCCAGCCTTAGTTAAAACTTTCGCAGCTCATATCAAAGATACTCTA
AACAGATTCAAAACTGAAAATGGACTGACTGACACAAAAGACGTCGTCCTCCAATTCAGTGCTCA
TTCTTTACCAATGGATATTGTCAATAAAGGAGATTCGTATCCTGCAGAAGTCGCAGCGAGTGTCT
TTGCCATTATGAAAGAACTTAACTTCTCAAATCCTTATAAATTAACCTGGCAATCACAGGTTGGC
CCAAAGCCTTGGCTGGGTGCTCAAACTGAAAAAATTACCAAGCAGCTAGCATCCAGTGATGTTCC
TGGAGTCGTTTTGGTTCCTATTGCCTTTACCTCTGATCATATTGAAACTCTCCATGAACTGGATA
TTGAACTGATTCAAGAACTACCTAATCCTTCAAAAGTAAAGCGAGTTGAATCGTTGAACGGAGAC
CAAACTTTCATTGACTCCTTGGCAGAACTAGTGAAGAGTCACATTGATTCGAAGGTTGTATTTTC
CAACCAGTTGCCATTGGATTCCATGCTGGGAGTAGTGTCAGATAATTCCCTCACAGATCCAAAAG
AGTTTTTCAGAGCCCATTGA

### Part C. Results and Discussion

### Example 24—Characterization of Strain MXY0183

Optimum growth conditions for Strain MXY0183 include a target pH of 3.0 to 6.0 and temperatures of 28-35° C. In order to produce the LegH protein, strain MXY0183 must be alive and growing aerobically for a period of 6 days.

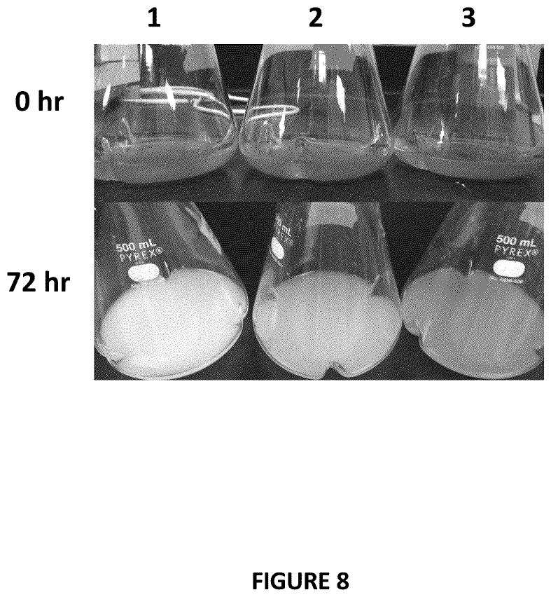

Expression of the genes associated with strain MXY0183 resulted in phenotypic changes to the strain. FIG. 8 shows photographs of shake flasks at the start of induction (0 hr) and 72 hr post-induction. The flasks designated #1 contain the host strain, MXY0051. The flasks designated #2 and #3 contain one of the intermediate strains (i.e., MXY0118, containing >10 copies of the LegH gene and the ALA dehydratase from the heme biosynthetic pathway) and the production strain (i.e., MXY0183, containing >10 copies of the LegH gene and the 8 enzymes from the heme biosynthetic pathway), respectively. The characteristic red color in flask #3 after 72 hours demonstrates the production of heme-bound LegH.

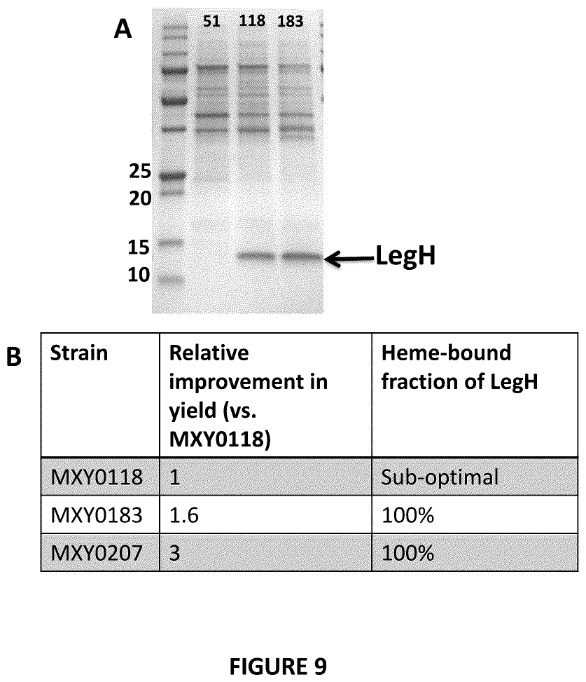

After growing in shake flasks, the P. pastoris strains indicated above, MXY0051, MXY0118, and MXY0183, were lysed and the proteins run on a SDS gel (FIG. 9A). The arrow shows the position of the LegH protein. A comparison of LegH production in strain MXY0183 and in strain MXY0118 is shown in FIG. 9B, which demonstrates the efficiency of heme loading of the LegH protein by the MXY0183 strain.

### Example 25—Characterization of Strain MXY0207

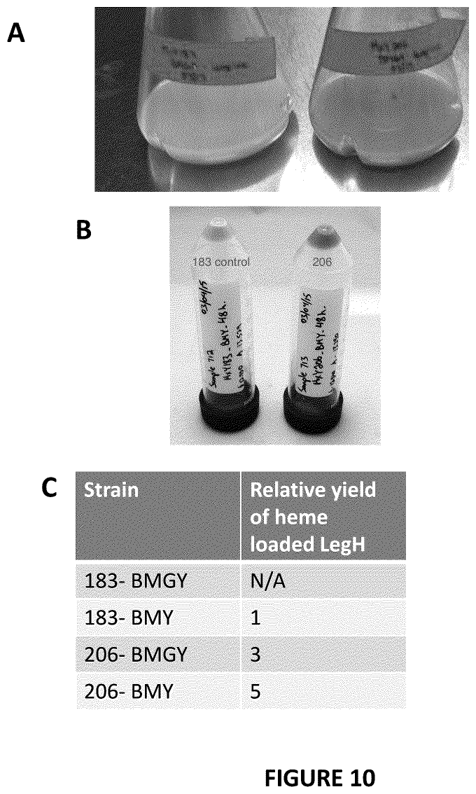

Experiments were then performed to determine the benefits of overexpressing the transcriptional activator, Mxr1, in the presence of the genes encoding the 8 enzymes involved in heme biosynthesis. Strain MXY0183, which contains >10 copies of the LegH sequence and the genes encoding the 8 enzymes involved in heme biosynthesis, and sister strains MXY0206 and MXY0207, which contain >10 copies of the LegH sequence, the genes encoding the 8 enzymes involved in heme biosynthesis, and the Mxr1 transcriptional activator, were grown in shake flask cultures in the presence of glycerol, which is a repressing carbon source for these strains. Photographs of the shake flask cultures after 48 hr are shown in FIG. 10A, and photographs of the pellets from cells grown on BMY media for 48 hours with no additional source of carbon are shown in FIG. 10B; these experiments demonstrated that significant expression of transgenes (e.g. heme enzymes) under the control of the AOX1 promoter occurs in the absence of an inducing carbon source when a repressing carbon source is consumed in the growth medium of a strain in which Mxr1 is also expressed from the AOX1 promoter. The relative yield of heme-loaded LegH, when shake flask cultures were grown in the absence of induction agent, is shown in FIG. 10C. These experiments demonstrated that significant production of a recombinant, heme-loaded protein is accomplished from AOX1 promoter-driven transgenes in the absence of methanol induction in Pichia strains in which Mxr1 expression is also driven by the AOX1 promoter.

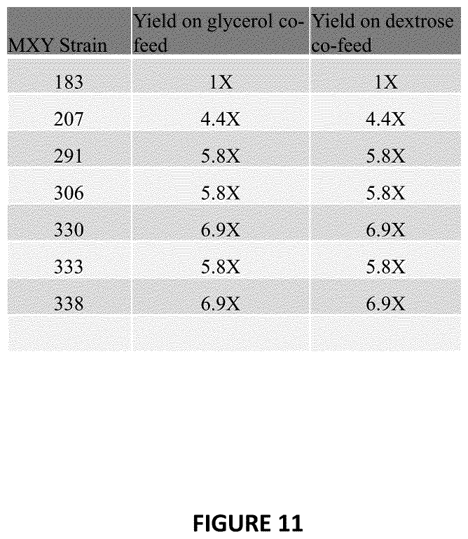

Select strains were grown in 2 L fermenter tanks, and the relative yield of LegH and heme-loaded LegH was determined (FIG. 11). Compared to strain MXY0183, the MXY0207 strain produced even more LegH and was able to produce enough heme to heme-load the LegH protein very effectively.

### Example 26—Characterization of Strain MXY0291

As described above in Examples 18-20, strain MXY0291 was constructed to recapitulate the LegH production ability of MXY0207, while being free of antibiotic resistance genes. It was determined that strain MXY0291 contained ˜16 copies of LegH var3, Mxr1 and 7 of the 8 heme biosynthetic enzymes. When grown in 2 L fermenter tanks, this strain showed improved LegH yield compared to MXY0207. This improvement was seen both in induction media containing methanol/glycerol and methanol/dextrose (D-glucose) (FIG. 11).

### Example 27—Characterization of Hybrid Promoter Strains

Additional copies of soybean leghemoglobin (LegH) were expressed under three different constitutive promoters, pGAP, pGCW14 and pTEF1, in a strain that already contains several copies of LegH, all heme biosynthetic enzymes, and the transcriptional factor Mxr1 under control of the promoter, pAOX1 (referred to above as MXY0291). When induced by methanol in the presence of dextrose (i.e., D-glucose), the constitutive promoters and pAOX1 drive expression of LegH while only the pAOX1 promoter drives expression of the heme enzymes. This leads to further improvement in LegH yield compared to previous strain MXY0291 (FIG. 11).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. (canceled)

2. A method of producing a heme-containing protein, the method comprising:
fermenting a recombinant Pichia pastoris cell lacking a recombinant nucleic acid encoding an antibiotic resistance gene in a fermentation broth,
wherein the recombinant Pichia pastoris cell comprises:
one or more stably integrated recombinant nucleic acids encoding one or more heme biosynthesis polypeptides selected from ALA synthase, ALA dehydratase, porphobilinogen deaminase, uroporphyrinogen III synthase, uroporphyrinogen III decarboxylase, coproporphyrinogen oxidase, protoporphyrinogen III oxidase, and ferrochelatase,
a stably integrated recombinant nucleic acid encoding the heme-containing protein operably linked to a first methanol-inducible promoter, and
a recombinant nucleic acid encoding a transcriptional activator.

3. The method of claim 2, wherein at least one of the one or more stably integrated recombinant nucleic acids encoding the one or more heme biosynthesis polypeptides is operably linked to a second methanol-inducible promoter.

4. The method of claim 2, wherein the recombinant nucleic acid encoding the transcriptional activator is operably linked to the first methanol-inducible promoter.

5. The method of claim 2, wherein the heme-containing protein is leghemoglobin.

6. The method of claim 2, further comprising purifying the heme-containing protein.

7. The method of claim 2, wherein the recombinant nucleic acid encoding the transcriptional activator is operably linked to a constitutive promoter.

8. The method of claim 2, wherein the recombinant nucleic acid encoding the transcriptional activator is operably linked to a third methanol-inducible promoter.

9. The method of claim 3, wherein the recombinant nucleic acid encoding the transcriptional activator is operably linked to a third methanol-inducible promoter.

10. The method of claim 2, wherein each of the one or more stably integrated recombinant nucleic acids encoding one or more heme biosynthesis polypeptides, the stably integrated recombinant nucleic acid encoding the heme-containing protein, and the recombinant nucleic acid encoding the transcriptional activator is independently operably linked to a Pichia pastoris promoter.

11. The method of claim 2, wherein the one or more stably integrated recombinant nucleic acids encoding one or more heme biosynthesis polypeptides, the stably integrated recombinant nucleic acid encoding the heme-containing protein, and the recombinant nucleic acid encoding the transcriptional activator are operably linked to a Pichia pastoris promoter.

12. The method of claim 2, wherein the one or more heme biosynthesis polypeptides are Pichia pastoris heme biosynthesis polypeptides.

13. The method of claim 2, wherein the recombinant Pichia pastoris cell comprises:
one or more stably integrated recombinant nucleic acids encoding ALA synthase, ALA dehydratase, porphobilinogen deaminase, uroporphyrinogen III synthase, uroporphyrinogen III decarboxylase, coproporphyrinogen oxidase, protoporphyrinogen III oxidase, and ferrochelatase.

14. The method of claim 2, wherein the transcriptional activator is a Pichia pastoris transcriptional activator.

15. The method of claim 2, wherein the transcriptional activator is selected from methanol expression regulator 1 (Mxr1) from Pichia pastoris, alcohol dehydrogenase regulator 1 (Adr1) from Hansemdla polymorpha, transcriptional regulation of methanol induction 1 (Trm1) from Candida boidinii, and transcriptional regulation of methanol induction 2 (Trm2) from Candida boidinii.

16. The method of claim 2, wherein the recombinant nucleic acid encoding the transcriptional activator is stably integrated.

17. The method of claim 2, further comprising supplementing the fermentation broth with iron or a pharmaceutically or metabolically acceptable salt thereof.

18. The method of claim 2, further comprising lysing the recombinant Pichia pastoris cell and purifying the heme-containing protein from the lysed recombinant Pichia pastoris cell.

19. The method of claim 2, wherein the heme-containing protein is a globin.

20. The method of claim 2, wherein the heme-containing protein is selected from a protease, a catalase, a peroxidase, an oxidoreductase, and a plant hemoglobin.

21. The method of claim 2, wherein the first methanol-inducible promoter is selected from alcohol oxidase 1 (AOX1), methanol oxidase (MOX), alcohol oxidase (AOD1), methanolica alcohol oxidase 1 (MOD1), methanolica alcohol oxidase 2 (MOD2), dihydroxyacetone synthase (DHAS), and peroxin 8 (PEX8).

22. The method of claim 15, wherein the first methanol-inducible promoter is selected from alcohol oxidase 1 (AOX1), methanol oxidase (MOX), alcohol oxidase (AOD1), methanolica alcohol oxidase 1 (MOD1), methanolica alcohol oxidase 2 (MOD2), dihydroxyacetone synthase (DHAS), and peroxin 8 (PEX8).

23. The method of claim 22, further comprising lysing the recombinant Pichia pastoris cell, and purifying the heme-containing protein from the lysed recombinant Pichia pastoris cell.

24. The method of claim 2, wherein the first methanol-inducible promoter is alcohol oxidase 1 (AOX1) and the transcriptional activator is methanol expression regulator 1 (Mxr1).

25. The method of claim 24, further comprising lysing the recombinant Pichia pastoris cell, and purifying the heme-containing protein from the lysed recombinant Pichia pastoris cell.

26. A method of producing a heme-containing protein, the method comprising:
fermenting a recombinant Pichia pastoris cell lacking a recombinant nucleic acid encoding an antibiotic resistance gene in a fermentation broth,
wherein the recombinant Pichia pastoris cell comprises:
a stably integrated recombinant nucleic acid encoding ALA synthase, operably linked to a first methanol-inducible promoter,
a stably integrated recombinant nucleic acid encoding ALA dehydratase, operably linked to a second methanol-inducible promoter,
a stably integrated recombinant nucleic acid encoding porphobilinogen deaminase, operably linked to a third methanol-inducible promoter,
a stably integrated recombinant nucleic acid encoding uroporphyrinogen III synthase, operably linked to a fourth methanol-inducible promoter,
a stably integrated recombinant nucleic acid encoding uroporphyrinogen III decarboxylase, operably linked to a fifth methanol-inducible promoter,
a stably integrated recombinant nucleic acid encoding coproporphyrinogen oxidase, operably linked to a sixth methanol-inducible promoter,
a stably integrated recombinant nucleic acid encoding protoporphyrinogen III oxidase, operably linked to a seventh methanol-inducible promoter,
a stably integrated recombinant nucleic acid encoding ferrochelatase, operably linked to a eighth methanol-inducible promoter,
two or more copies of a stably integrated recombinant nucleic acid encoding the heme-containing protein, operably linked to a ninth methanol-inducible promoter, and
a recombinant nucleic acid encoding a transcriptional activator operably linked to a constitutive promoter or a tenth methanol-inducible promoter,
wherein each of the first through ninth methanol-inducible promoters comprises a sequence to which the transcriptional activator binds.
